(19) United States
(12) Patent Application Publication
(10) Pub. No.: US 2022/0161243 A1
(43) Pub. Date: May 26, 2022

(54) Catalyst Compositions and Precursors, Processes for Making the Same and Syngas Conversion Processes

(71) Applicant: ExxonMobil Chemical Patents Inc., Baytown, TX (US)

(72) Inventors: Jeffrey C. Bunquin, Houston, TX (US); Partha Nandi, Annandale, NJ (US); Paul F. Keusenkothen, Houston, TX (US); Vera Grankina, Doylestown, PA (US); Robert Panepinto, Freehold, NJ (US)

(21) Appl. No.: 17/433,641

(22) PCT Filed: Feb. 6, 2020
(86) PCT No.: PCT/US2020/016938

§ 371 Date: Aug. 25, 2021

Related U.S. Application Data
(60) Provisional application No. 62/811,836, filed on Feb. 28, 2019.

(30) Foreign Application Priority Data
Jun. 13, 2019 (EP) 19179929.5

(51) Int. Cl. B01J 31/18; B01J 37/08; B01J 37/02; C07C 1/04; C10G 2/00

(52) U.S. Cl. CPC B01J 31/1805; C07C 2523/83; B01J 37/0201; C07C 1/043; C07C 1/0435; C10G 2/332; B01J 2531/845; B01J 2531/37; B01J 2531/72; C07C 2531/22; C07C 2523/10; C07C 2523/34; C07C 2523/75; C07C 2523/889; B01J 37/08

(57) ABSTRACT

Disclosed are novel catalyst compositions, catalyst precursors, processes for making catalyst precursors, processes for making catalyst compositions, and processes for converting syngas. The catalytic component in the catalyst composition can comprise a metal carbide and/or a metal nitride. This disclosure is particularly useful for converting syngas via the Fischer-Tropsch reactions to make olefins and/or alcohols.

### PRIORITY

This application claims priority to U.S. Provisional Application No. 62/811,836, filed Feb. 28, 2019, and EP Search Report Application No. 19179929.5 filed Jun. 13, 2019, the disclosures of which are incorporated in their entirety.

### FIELD

The present disclosure relates to catalyst compositions, catalyst precursors, processes for making catalyst precursors, processes for making catalyst compositions, and processes for converting syngas. This disclosure is useful, e.g., in converting syngas into olefins and/or alcohols, especially C2-05 olefins and Cl-05 alcohols.

### BACKGROUND

Synthesis gas (syngas) is a mixture of hydrogen and carbon monoxide generated from the upgrading of chemical feedstocks such as natural gas and coal. Syngas has been used industrially for the production of value-added chemicals including chemical intermediates such as olefins and alcohols, and fuels. Fischer-Tropsch catalysis is one route for syngas conversion to value-added products. Generally, Fischer-Tropsch catalysis involves the use of iron and cobalt catalysts for the production of gasoline range products for transportation fuels, heavy organic products including distillates used in diesel fuels, and high purity wax for a range of applications including food production.

Similar catalysts can be used for the production of value-added chemical intermediates including olefins and alcohols that can be used, for example, for the production of polymers and fuels. Often the production of value-added chemicals includes the production of saturated hydrocarbons including paraffins. The selectivity of Fischer-Tropsch catalysts towards production of value-added chemical intermediates may be adjusted by addition of promoters including group 1 and group 2 cations and transition metals. Fischer-Tropsch catalysts have been prepared as metal oxides or sulfides of iron and cobalt. The iron and cobalt catalysts are frequently supported on solid carriers including oxides such as alumina, silica, or various clays or on carbonaceous materials. Fischer-Tropsch catalysts have been used to produce hydrocarbons in the gasoline range and lighter hydrocarbons.

When used to produce light hydrocarbons (C1-C5), the selectivity of conventional Fischer-Tropsch catalysts towards value-added chemicals over methane can be low. Metal nitrides and metal carbides are sought-after materials for a variety of applications. Metal nitrides and carbides have useful applications in areas other than catalysis. For example, Xiao et al, ACS Nano, 2014, 8, 7846-7857, discloses that transition metal carbides show an unusual combination of outstanding properties, such as high melting point, high electrical and thermal conductivities, exceptional hardness, excellent mechanical stability, and chemical stability along with high corrosion resistance under reaction conditions.

Generally, synthetic methods for making nitrides involve a high temperature reaction (typically 650° C. or above) of metal precursors with ammonia via gas-solid reactions or by vapor deposition of metal salt precursors. See Wriedt, Bull, of Alloy Phase Diagr., 1989, 10(4), 358-67 (methods of making tungsten nitrides by contacting tungsten films with ammonia at high temperatures); Nandi et al. ACS Appl. Mater. Interfaces, 2014, 6, 6606-6615 (atomic layer deposition of molybdenum nitride films); Chem. Mater., 2003, 15, 2969-2976, WO00/41404 (Gelest) and Phys. Chem. Chem. Phys., 2015, 17, 17445-17453 (atomic layer deposition of tungsten nitride films); and Polcar et al., Wear, 2007, 262, 655-665 (deposition of tungsten nitride coatings by reactive magnetron sputtering). Such methods typically provide materials with a relatively low surface area and the harsh conditions prevent accurate control of particle size. Furthermore, it is often difficult or impossible to make mixed metal nitrides by such methods. Porous Co₃ZnC nanoparticles (a mixed metal carbide) was synthesized by annealing Zn₃[Co(CN)₆]₂/polyvinylpyrrolidone nano-sphere precursors under a nitrogen atmosphere at a temperature of 600° C. Xiao et al, ACS Nano, 2014, 8, 7846-7857. One method of making iron carbide is by thermal decomposition of “Prussian Blue” (Fe₄[Fe(CN)₃). Aparicio et al., J. Therm. Anal. Calorim. 2012, 110, 661; Zakaria et al., RSC Adv., 2016, 6, 10341). Certain metal nitrides have been prepared at low temperature with the use of supercritical ammonia (e.g. J. Mater. Chem., 2004, 14, 228-32), but the process requires a very high pressure and specialized equipment.

There remains a need for improved catalyst compositions for use in syngas conversion, particularly Fischer-Tropsch conversion of syngas into light alcohols and olefins with low methane formation. Furthermore, there remains a need for a convenient route to making metal nitrides and carbides and mixed metal nitrides and carbides from readily available starting materials.

References for citing in an information disclosure statement (37 C.F.R 1.97(h)): U.S. Pat. Nos. 10,022,712; 9,416,067; U.S. Patent Publication Nos. 2017/051054, and 2002/0010221.

### BRIEF DESCRIPTION OF THE DRAWING

FIG. 1 is a graph showing X-ray diffraction (“XRD”) patterns of 7 bimetallic, iron-containing catalyst precursors of this disclosure.

FIG. 2 is a graph showing XRD patterns of 3 trimetallic, cobalt-containing catalyst precursors of this disclosure.

FIG. 3 is a graph showing XRD patterns of 6 used catalytic components of this disclosure.

FIG. 4 is a graph showing the thermogravimetric analysis results of an iron-containing catalyst precursor of this disclosure.

FIG. 5 is a graph showing thermogravimetric analysis result of a cobalt-containing catalyst precursor of this disclosure.

FIG. 6 is a graph showing an XRD pattern of a cobalt-containing catalytic component of this disclosure, and three peak groups identified therein corresponding to three different phases.

FIG. 7 is a graph showing the XRD pattern of the catalytic component shown in FIG. 7, and seven additional peak groups identified therein corresponding to seven additional phases.

FIG. 8 is a graph showing the XRD pattern of the catalytic component shown in FIGS. 6 and 7, and three additional peak groups identified therein corresponding to three additional phases.

FIG. 9 is a graph comparing activity in terms of CO conversion as a function of time on stream (“TOS”) of an inventive, trimetallic Co-La-Mn-containing, and metal carbide/nitride-containing catalyst composition, and a comparative, trimetallic Co-La-Mn-oxide-containing catalyst composition substantially free of a metal carbide/nitride.

FIGS. 10 and 11 are graphs showing C2-C4 alcohol selectivity and C5-C11 alcohol selectivity as a function of CO conversion, respectively, of an exemplary syngas conversion process utilizing an exemplary, trimetallic Co-Y-Mn-containing, metal carbide/nitride-containing catalyst composition of this disclosure.

### SUMMARY

It has been found, in a surprising manner, that catalytic components highly active for converting syngas comprising two, three, or more metals, at least partly in metal carbide(s) and/or metal nitride(s) phases, can be fabricated by thermally decomposing a catalyst precursor comprising a complex salt or ionic network of the metals at mild temperatures much lower than the traditional processes for making metal carbides and nitrides. The metal carbide(s) and/or metal nitride(s) phases in the catalytic component are highly dispersed in the catalytic component, leading to high catalyst activity.

A first aspect of this disclosure relates to a catalyst composition such as a catalyst composition for converting syngas comprising a catalytic component, wherein the catalytic component comprises: a metal element M¹, selected from iron, cobalt, manganese, and combinations of two or more thereof at any proportion; a metal element M², selected from aluminum, gallium, indium, thallium, chromium, scandium, yttrium, the lanthanide series, the actinide series, and any combination of two or more thereof at any proportion; an optional metal M³, differing from M¹ and M²; carbon; nitrogen; and optionally sulfur, at a molar ratio of M², M³, carbon, nitrogen, and sulfur to M¹ of r1, r2, r3, r4, and r5, respectively, indicated below: M²:M³:C:N:S:M¹=r1:r2T3T4T5:1, where: 0.1≤r1≤1.5; 0≤r2≤0.5; 0≤r3≤1; 0≤r4≤1; and 0≤r5≤1.

A second aspect of this disclosure relates to a catalyst composition comprising a catalytic component, wherein the catalytic component comprises: a metal element M¹, selected from iron, cobalt, manganese, and combinations of two or more thereof at any proportion; a metal element M², selected from aluminum, gallium, indium, thallium, chromium, scandium, yttrium, the lanthanide series, the actinide series, and any combination of two or more thereof at any proportion; an optional metal M³, differing from M¹ and M²; carbon; nitrogen; and optionally sulfur, and wherein: at least a portion of the carbon in the catalytic component is present as a metal carbide of one or more of M¹, M², and M³, and at least a portion of the nitrogen in the catalytic component is present as a metal nitride of one or more of M¹, M², and M³, as determined by x-ray diffraction diagram of the catalytic component.

A third aspect of this disclosure relates to a catalyst precursor of a catalyst, comprising a first precursor component having the following formula (F-PM-1), a second precursor component having the following formula (F-PM-2), or a mixture or combination of both the first precursor component and the second precursor component:

M^{b}_{q-p}(M^{a}L_{q})ₘ (F-PM-1)

M^{b}Lⱼ (F-PM-2)

where M^{a} is a metal element in +p valency selected from manganese, iron, cobalt, and combinations and mixtures of two or more thereof at any proportion, L, the same or different at each occurrence, is a ligand selected from CN⁻, OCN⁻, and SCN⁻, M^{a} complexes with q units of L on average to form a complex anion in p-q average valency, M^{b} is a metal element selected from aluminum, gallium, indium, thallium, iron, cobalt, chromium, manganese, manganese, scandium, yttrium, the lanthanide series, the actinide series, and any combination or mixture of two or more thereof at any proportion, providing a cation in +m valency, where j is an integer or non-integer, and m−1≤j≤m, m is 2, 3, 4, 5, or 6, p is 2, 3, 4, or 5, q is an integer or non-integer, and 2≤q≤6.

A fourth aspect of this disclosure relates to a process for making a catalytic composition, the process comprising:

(i) providing a first material comprising a first compound having the following formula (F-I-1), and/or a second compound having the following formula (F-1-2), or a mixture of the first compound and the second compound:

M^{d}_{q-p}(M^{a}L_{q})ₖ (F-I-1)

M^{e}Lₓ (F-I-2)

where M^{a} is a metal element in +p valency selected from manganese, iron, cobalt, and combinations and mixtures of two or more thereof at any proportion, L, the same or different at each occurrence, is a ligand selected from CN⁻, OCN⁻, and SCN⁻, M^{a} complexes with q units of L on average to form a complex anion in p-q average valency, M^{d} is a metal element or a group providing a cation in +k valency, and M^{e} is a metal element or a group providing a cation in +x valency, where p is 2, 3, 4, or 5, q is an integer or non-integer, 2≤q≤6, k is 1, 2, 3, 4, 5, or 6, and x is 1, 2, 3, 4, 5, or 6;

(ii) providing a second material having the following formula (F-II):

M^{b}ₙAₘ (F-II)

where M^{b} is a metal element in +m valency selected from aluminum, gallium, indium, thallium, iron, cobalt, chromium, manganese, manganese, scandium, yttrium, the lanthanide series, the actinide series, and any combination or mixture of two or more thereof at any proportion, and A is an anion in −n valency, wherein A differs from the complex anion in (F-I), m is 2, 3, 4, 5, or 6, and n is 1, 2, 3, 4, 5, or 6; and

(iii) reacting the first material and the second material to obtain a first solid precursor comprising first precursor component having the following formula (F-PM-1), or a second precursor component having the following formula (F-PM-2), or a mixture or combination of both the first precursor component and the second precursor component:

M^{b}_{q-p}(M^{a}L_{q})ₘ (F-PM-1)

M^{b}Lⱼ (F-PM-2)

where j is an integer or non-integer, and m−1≤j≤m.

A fifth aspect of this disclosure relates to a process for converting syngas, the process comprising contacting a feed comprising syngas with a catalyst composition of the first aspect described summarily above in a conversion reactor to produce a conversion product mixture.

A sixth aspect of this disclosure relates to a process for converting syngas, the process comprising contacting a feed comprising syngas with a catalyst composition of the second aspect described summarily above in a conversion reactor to produce a conversion product mixture.

A seventh aspect of this disclosure relates to a process for converting syngas, the process comprising: (A) disposing a catalyst precursor of the second aspect described summarily above in a conversion reactor; (B) heating the catalyst precursor in the conversion reactor at a temperature of at least 200° C. in the presence of an inert atmosphere for a period of at least 1 minute to obtain a catalytic component; and (C) contacting the catalytic component with a feed comprising syngas under conversion conditions effective to convert syngas to a conversion product mixture.

### DETAILED DESCRIPTION

In this disclosure, a process is described as comprising at least one “step.” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other step, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are conducted in the order described.

Unless otherwise indicated, all numbers indicating quantities in this disclosure are to be understood as being modified by the term “about” in all instances. It should also be understood that the numerical values used in the specification and claims constitute specific embodiments. Efforts have been made to ensure the accuracy of the data in the examples. However, it should be understood that any measured data inherently contain a certain level of error due to the limitation of the technique and equipment used for making the measurement.

As used herein, the indefinite article “a” or “an” shall mean “at least one” unless specified to the contrary or the context clearly indicates otherwise. Thus, embodiments comprising “a metal” include embodiments comprising one, two, or more metals, unless specified to the contrary or the context clearly indicates only one metal is included.

For the purposes of this disclosure, the nomenclature of elements is pursuant to the version of Periodic Table of Elements as described in CHEMICAL AND ENGINEERING NEWS, 63(5), pg. 27 (1985). Abbreviations for atoms are as given in the periodic table (Li=lithium, for example).

The following abbreviations may be used herein for the sake of brevity: RT is room temperature (and is 23° C. unless otherwise indicated), kPag is kilopascal gauge, psig is pound-force per square inch gauge, psia is pound-force per square inch absolute, and WHSV is weight hourly space velocity, and GHSV is gas hourly space velocity.

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of this disclosure. Additionally, they do not exclude impurities and variances normally associated with the elements and materials used. “Consisting essentially of” a component in this disclosure can mean, e.g., comprising, by weight, at least 80 wt %, of the given material, based on the total weight of the composition comprising the component.

“Soluble” means, with respect to a given solute in a given solvent at a given temperature, at most 100 mass parts of the solvent is required to dissolve 1 mass part of the solute under a pressure of 1 atmosphere. “Insoluble” means, with respect to a given solute in a given solvent at a given temperature, more than 100 mass parts of the solvent is required to dissolve 1 mass part of the solute under a pressure of 1 atmosphere.

The term “Cn” compound or group, where n is a positive integer, means a compound or a group comprising carbon atoms therein at the number of n. Thus, “Cm to Cn” alcohols means an alcohol comprising carbon atoms therein at a number in a range from m to n, or a mixture of such alcohols. Thus, C1-C2 alcohols means methanol, ethanol, or mixtures thereof.

The term “conversion” refers to the degree to which a given reactant in a particular reaction (e.g., dehydrogenation, hydrogenation, etc.) is converted to products. Thus 100% conversion of carbon monoxide means complete consumption of carbon monoxide, and 0% conversion of carbon monoxide means no measurable reaction of carbon monoxide.

The term “selectivity” refers to the degree to which a particular reaction forms a specific product, rather than another product. For example, for the conversion of syngas, 50% selectivity for C1-C4 alcohols means that 50% of the products formed are C1-C4 alcohols, and 100% selectivity for C1-C4 alcohols means that 100% of the products formed are C1-C4 alcohols. The selectivity is based on the product formed, regardless of the conversion of the particular reaction. The selectivity for a given product produced from a given reactant can be defined as weight percent (wt %) of that product relative to the total weight of the products formed from the given reactant in the reaction.

Detailed description of the catalyst compositions of this disclosure, including the catalyst composition of the first aspect and the catalyst composition of the second aspect of this disclosure, the catalyst precursor, the process for making a catalyst composition, and the processes for converting syngas utilizing a catalyst composition or a catalyst precursor, is provided below. In the description, unless specified or the context clearly indicates otherwise, a “catalyst composition of this disclosure” means a catalyst composition of the first aspect of this disclosure, a catalyst composition of the second aspect of this disclosure, or a mixture or a combination thereof.

### Catalyst Compositions

In the catalyst compositions of this disclosure, preferably M¹ is selected from iron, cobalt, combinations of iron and cobalt at any proportion, combinations of iron and manganese at any proportion, combination of cobalt with manganese at any proportion, and combination of iron, cobalt, and manganese at any proportion. In specific embodiments, M¹ is a single metal of cobalt or iron. Where M¹ comprises a binary mixture/combination of cobalt and manganese, preferably cobalt is present at a higher molar proportion than manganese. Where M¹ comprises a binary mixture/combination of iron and manganese, preferably iron is present at a higher molar proportion than manganese. Without intending to be bound by a particular theory, it is believed that the presence of M¹ provides at least a portion of the catalytic effect of the catalytic component of the catalyst composition of the first aspect of this disclosure.

Preferably M² is selected from aluminum, gallium, indium, thallium, chromium, scandium, yttrium, and the lanthanide series. More preferably M² is selected from gallium, indium, scandium, yttrium, and the lanthanide series. Particularly desirable lanthanide series for the catalyst composition of the first aspect of this disclosure include, but are not limited to: La, Ce, Pr, Nd, Gb, Dy, Ho, and Er. Without intending to be bound by a particular theory, it is believed that the presence of M² promotes the catalytic effect of M¹ in the catalyst compositions of this disclosure.

The presence of M³ in the catalyst compositions of this disclosure is optional. If present, M³ is preferably selected from alkali metals, copper, silver, and any combinations and mixtures of two or more thereof at any proportion. In certain embodiments, M³ is selected from copper, silver, and mixtures/combinations thereof. Without intending to be bound by a particular theory, it is believed the presence of metal M³ can promote the catalyst effect of the catalyst compositions of this disclosure.

Metal carbides such as iron carbide and cobalt carbide have been reported as catalysts for converting syngas to make various organic compounds. The catalyst compositions of this disclosure comprise a catalytic component comprising carbon. It is believed that in the catalytic component of a catalyst composition of this disclosure, carbon may be present at least in part as a carbide of a metal. The presence of a metal carbide can be indicated by the XRD graph of the catalyst composition. By a “metal carbide,” it is meant to include carbide of a single metal, or a combination of two or more metals M¹, M², and/or M³. Desirably the catalytic component comprises a carbide of a single metal, or a combination of two or more metals of M¹, and/or M². Desirably the catalytic component comprises a carbide of a single metal, or a combination of two or more metals of M¹. Desirably the catalytic component comprises one or more of iron carbide, cobalt carbide, manganese carbide, (mixed iron cobalt) carbide, (mixed iron manganese) carbide, mixed (cobalt manganese) carbide, and mixed (cobalt, iron, and manganese) carbide. Desirably, the catalytic component comprises a carbide of a single metal, or a combination of two or more metals of M² (e.g., yttrium and the lanthanides). The catalytic component may comprise a carbide of a metal mixture comprising an M¹ and an M². The identification of the presence of a carbide phase in a catalyst composition can be conducted by comparing the XRD data of the catalyst composition against an XRD peak database of known carbides, such as those available from International Center for Diffraction Data (“ICDD”).

A novel feature of the catalyst compositions for converting syngas of the first aspect of this disclosure resides in the presence of nitrogen in the catalytic component of the of the catalyst composition, in addition to carbon. It is believed that in the catalytic component of the catalyst composition of this disclosure, nitrogen may be present in part as a nitride of a metal. The presence of a metal nitride can be indicated by the XRD graph of the catalyst composition. By a “metal nitride,” it is meant to include nitride of a single metal, or a combination of two or more metals of M¹, M², and M³. Desirably the catalytic component comprises a nitride of a single metal, or a combination of two or more metals of M¹, and/or M². Desirably the catalytic component comprises a nitride of a single metal, or a combination of two or more metals of M¹. Desirably the catalyst comprises one or more of iron nitride, cobalt nitride, manganese nitride, (mixed iron cobalt) nitride, (mixed iron manganese) nitride, mixed (cobalt manganese) nitride, and mixed (cobalt, iron, and manganese) nitride. Desirably, the catalytic component comprises a nitride of a single metal, or a combination of two or more metals of M² (e.g., yttrium and the lanthanides). The catalytic component may comprise a nitride of a metal mixture comprising an M¹ and an M². The identification of the presence of a nitride phase in a catalyst composition can be conducted by comparing the XRD data of the catalyst composition against an XRD peak database of known nitrides.

The catalyst compositions of this disclosure may optionally comprise sulfur in the catalytic component thereof. Without intending to be bound by a particular theory, in certain embodiments, the presence of sulfur can promote the catalytic effect of the catalyst composition. The sulfur may be present as a sulfide of one or more metals of M¹, M², and/or M³.

In specific embodiments, the catalytic component of a catalyst composition of this disclosure consists essentially of M¹, M², M³, carbon, nitrogen, and optionally sulfur, e.g., comprising ≥85, or ≥90, or ≥95, or ≥98, or even ≥99 wt % of M¹, M², M³, carbon, nitrogen, and optionally sulfur, based on the total weight of the catalytic component.

The molar ratios of M², M³, carbon, nitrogen, and sulfur to M¹, r1, r2, r3, r4, and r5, respectively, in the catalytic component of a catalyst composition of this disclosure are calculated from the aggregate molar amounts of the elements in question. Thus, if M¹ is a combination/mixture of two or more metals, the aggregate molar amounts of all metals M¹ is used for calculating the ratios. If M² is a combination/mixture of two or more metals, the aggregate molar amounts of all metals M² is used for calculating the ratio r1. If M³ is a combination/mixture of two or more metals, the aggregate molar amounts of all metals M³ is used for calculating the ratio r2.

Preferably, the molar ratio of M² to M¹ in the catalytic component of a catalyst compositions of this disclosure, r1, can range from r1a to r1b, where r1a and r1b can be, independently, e.g., 0.1, 0.2, 0.3, 0.4, 0.5, 0.6, 0.7, 0.8, 0.9, 1.0, 1.1, 1.2, 1.3, 1.4 or 1.5, as long r1a<r1b. More preferably r1a=0.8, r1b=1.2; still more preferably r1a=0.9, r1b=1.1. In one particularly advantageous embodiment, r1 is in the vicinity of 1.0 (e.g., from 0.95 to 1.05), meaning that M¹ and M² are present in the catalytic component at substantially equivalent molar amounts.

Preferably, the molar ratio of M³ to M¹ in the catalytic component of a catalyst compositions of this disclosure, r2, can range from r2a to r2b, where r2a and r2b can be, independently, e.g., 0, 0.1, 0.2, 0.3, 0.4, or 0.5, as long as r2a<r2b. Thus M³, if present, is at a substantially lower molar amount than M¹.

Preferably, the molar ratio of carbon to M¹ in the catalytic component of a catalyst composition of this disclosure, r3, can range from r3a to r3b, where r3a and r3b can be, independently, e.g., 0.1, 0.2, 0.3, 0.4, 0.5, 0.6, 0.7, 0.8, 0.9, or 1.0, as long as r3a<r3b.

Preferably, the molar ratio of nitrogen to M¹ in the catalytic component of a catalyst composition of this disclosure, r4, can range from r4a to r4b, wherein r4a and r4b can be, independently, e.g., 0.1, 0.2, 0.3, 0.4, 0.5, 0.6, 0.7, 0.8, 0.9, or 1.0, as long as r4a≤r4b.

Preferably, the molar ratio of sulfur to M¹ in the catalytic component of a catalyst composition of this disclosure, r5, can range from r5a to r5b, wherein r5a and r5b can be, independently, e.g., 0, 0.1, 0.2, 0.3, 0.4, 0.5, 0.6, 0.7, 0.8, 0.9, 1.0, as long as r5a<r5b. Preferably r5a=0, and r5b=0.5. Still more preferably r5a=0 and r5b=0.3.

In specific embodiments, the metal(s) M¹ can be distributed substantially homogeneously in the catalytic component. Additionally and/or alternatively, the metal(s) M² can be distributed substantially homogeneously in the catalytic component. Additionally and/or alternatively, carbon can be distributed substantially homogeneously in the catalytic component. Still additionally and/or alternatively, nitrogen can be distributed substantially homogeneously in the catalytic component.

It is highly advantageous that the metal carbide(s) and/or the metal nitride(s) are highly dispersed in the catalytic component. The metal carbide(s) and/or the metal nitride(s) can be substantially homogeneously distributed in the catalytic component, resulting in a highly dispersed distribution, which can contribute to a high catalytic activity of the catalytic component.

Without intending to be bound by a particular theory, it is believed that the metal carbide(s), the metal nitride(s), and possibly the elemental phases of M¹ in the catalytic component provide the desired catalytic activity for chemical conversion processes such as syngas conversion processes. One or more of M² and/or M³ can provide direct catalytic function as well. In addition, one or more of M² and/or M³ can perform the function of a “promoter” in the catalytic component. Furthermore, sulfur, if present, can perform the function of a promoter in the catalytic component as well. Promoters typically improve one or more performance properties of a catalyst. Example properties of catalytic performance enhanced by inclusion of a promoter in a catalyst over the catalyst composition without a promoter, may include selectivity, activity, stability, lifetime, regenerability, reducibility, and resistance to potential poisoning by impurities such as sulfur, nitrogen, and oxygen.

The catalyst composition of this disclosure may consist essentially of the catalytic component of this disclosure, e.g., comprising ≥85, or ≥90, or ≥95, or ≥98, or even ≥99 wt % of the catalytic component, based on the total weight of the catalyst composition. Such catalyst composition may be considered as a “bulk catalyst” in that it comprises minor amount of carrier or support material in its composition, if any at all. Bulk catalysts can be conveniently made by thermal decomposition from a catalyst precursor, as described below.

The catalyst composition of this disclosure can comprise a catalyst support material (which may be called a carrier or a binder), at any suitable quantity, e.g., ≥20, ≥30, ≥40, ≥50, ≥60, ≥70, ≥80, ≥90, or even ≥95 wt %, based on the total weight of the catalyst composition. In supported catalyst compositions, the catalytic component can be desirably disposed on the internal or external surfaces of the catalyst support material. Catalyst support materials may include porous materials that provide mechanical strength and a high surface area. Non-limiting examples of suitable support materials can include oxides (e.g. silica, alumina, titania, zirconia, and mixtures thereof), treated oxides (e.g. sulphated), crystalline microporous materials (e.g. zeolites), non-crystalline microporous materials, cationic clays or anionic clays (e.g. saponite, bentonite, kaoline, sepiolite, hydrotalcite), carbonaceous materials, or combinations and mixtures thereof. Deposition of the catalytic component on a support can be effected by, e.g., incipient impregnation. A support material can be sometimes called a binder in a catalyst composition.

### Catalyst Precursors

The catalytic component of the catalyst composition, or the catalyst composition per se, of this disclosure may be produced from a catalyst precursor. The catalyst precursor, which is another aspect of this disclosure, comprises a first precursor component having the following formula (F-PM-1), a second precursor component having the following formula (F-PM-2), or a mixture or combination of both the first precursor component and the second precursor component:

M^{b}_{q-p}(M^{a}L_{q})ₘ (F-PM-1)

M^{b}Lⱼ (F-PM-2)

where M^{a} is a metal element in +p valency selected from manganese, iron, cobalt, and combinations and mixtures of two or more thereof at any proportion, L, the same or different at each occurrence, is a ligand selected from CN⁻, OCN⁻, and SCN⁻, in (F-PM-1), M^{a} complexes with q units of L on average to form a complex anion in p-q average valency, M^{b} is a metal element selected from aluminum, gallium, indium, thallium, iron, cobalt, chromium, manganese, scandium, yttrium, the lanthanide series, the actinide series, and any combination or mixture of two or more thereof at any proportion, providing a cation in +m valency, where j is an integer or non-integer, and m−1≤j≤m, m is 2, 3, 4, 5, or 6, p is 2, 3, 4, or 5, q is an integer or non-integer, and 2≤q≤6.

In specific embodiments, the first precursor component having formula (F-PM-1) is a solid insoluble in deionized water at room temperature, and/or the second precursor component having formula (F-PM-2) is a solid insoluble in deionized water at room temperature. The solids of the first precursor component and/or the second precursor component may be present in the form of solid particles or solid in gels comprising the solid and solvent. For the purpose of this disclosure, a gel is regarded as a dispersion comprising insoluble solid.

In specific embodiments, the first precursor component having formula (F-PM-1) wherein m=q-p, in which case formula (F-PM-1) can be simplified as M^{b}(M^{a}L_{q}). In one example of such embodiments, m=q-p=3, q=6, and p=3, in which case formula (F-PM-1) can be simplified as M^{b}(M^{a}L₆). Specific, non-limiting examples of the first precursor component include: ME(III)[Fe(III)(CN)₆], ME(III)[Fe(III)(OCN)₆], ME(III)[Fe(III)(SCN)₆], ME(III)[Fe(II)(CN)₅], ME(III)[Fe(II)(OCN)₅], ME(III)[Fe(II)(SCN)₅], ME(III)[Co(III)(CN)₆], ME(III)[Co(III)(OCN)₆], ME(III)[Co(III)(SCN)₆], ME(III)[Co(II)(CN)₅], ME(III)[Co(II)(OCN)₅], and ME(III)[Co(II)(SCN)₅], where ME is a metal element selected from scandium, yttrium, cobalt, manganese, iron, aluminum, gallium, indium, the lanthanides, and the actinides.

The catalyst precursor comprising the first precursor component having formula (F-PM-1) and/or a second precursor component having formula (F-PM-2) may represent an ionic compound having formula (F-PM-1) wherein each M^{b} metal atom is bonded with q units of ligand L, which are not bonded with any other metal atom and/or an ionic compound having formula (F-PM-2) wherein each M^{a} and M^{b} metal atom is bonded with m units of ligands L, which are not bonded with any other metal atom.

The first precursor component having formula (F-PM-1) may represent an ionic network wherein one M^{a} metal atom is bonded with, on average, q units of ligands, at least some of which can be bonded with another metal atom. Such ligands capable of bonding with only one metal atom is called mono-dentate, capable of bonding with two metal atoms are called bidentate ligands, and such ligands capable of bonding with three metal atoms tridentate ligands. The number q in the formula (F-PM-1) representing an ionic network can be an integer or a non-integer. The ionic network is desirably insoluable in water at room temperature and one atmospheric pressure. Given that the CN⁻, OCN⁻ and SCN⁻ ligands are bidentate, such first precursor component comprising CN⁻, OCN⁻ and/or SCN⁻ ligands can form a network solid having a formula (F-PM-1) where the number q can be a non-integer instead of an integer. Such network solid can be dispersed in a solvent such as water to form a gel. Desirably the negative charges of the ligands in the network are balanced by the positive charges of the M^{a} cations, forming an electrically neutral network, which can be in the form of a gel dispersed in a solvent such as water.

Likewise, the second precursor component having formula (F-PM-2) may represent an ionic network wherein one M^{b} metal atom is bonded with, on average, j units of ligands, where j can be any number from m−1 to less than m. At least some of the bidentatate ligands can be bonded with another metal atom. Desirably the negative charges of the ligands in the network are balanced by the positive charges of the M^{b} cations, forming an electrically neutral network, which can be in the form of a gel dispersed in a solvent such as water.

A mixture of the first precursor component having formula (F-PM-1) and a second precursor component having formula (F-PM-2) can form an interconnected ionic network wherein M^{a} and M^{b} atoms are bonded, on average, q units of ligands and j units of ligands, respectively. Desirably the negative charges of the ligands in the network are balanced by the positive charges of the M^{a} and M^{b} cations, forming an electrically neutral network, which can be in the form of a gel dispersed in a solvent such as water.

It is possible that the ionic network described above present in the catalyst precursor may comprises M^{a} and M^{b} cations not completely balanced electrically with the ligands bonded with them in certain locations in the network. In such case, additional cations, such as alkali metal ions, an ammonium ion, a proton, and the like, may be entrained in the network to electrically balance the network.

### Processes for Making Catalyst Precursors

Another aspect of this disclosure relates to a process for making a catalyst precursor, such as a catalyst precursor as described above as an aspect of this disclosure. The process comprises:

(i) providing a first material comprising a first compound having the following formula (F-I-1), and/or a second compound having the following formula (F-1-2), or a mixture of the first compound and the second compound:

M^{d}_{q-p}(M^{a}L_{q})ₖ (F-I-1)

M^{e}Lₓ (F-I-2)

where M^{a} is a metal element in +p valency selected from manganese, iron, cobalt, and combinations and mixtures of two or more thereof at any proportion, L, the same or different at each occurrence, is a ligand selected from CN⁻, OCN⁻, and SCN⁻, M^{a} complexes with q units of L on average to form a complex anion in p-q average valency, M^{d} is a metal element or a group providing a cation in +k valency, and M^{e} is a metal element or a group providing a cation in +x valency, where p is 2, 3, 4, or 5, 2≤q≤6, k is 1, 2, 3, 4, 5, or 6, and x is 1, 2, 3, 4, 5, or 6;

(ii) providing a second material having the following formula (F-II):

M^{b}ₙAₘ (F-II)

where M^{b} is a metal element in +m valency selected from aluminum, gallium, indium, thallium, iron, cobalt, chromium, manganese, scandium, yttrium, the lanthanide series, the actinide series, and any combination or mixture of two or more thereof at any proportion, and A is an anion in −n valency, wherein A differs from the complex anion in (F-I-1), m is 2, 3, 4, 5, or 6, and n is 1, 2, 3, 4, 5, or 6; and

(iii) reacting the first material and the second material to obtain a first solid precursor comprising first precursor component having the following formula (F-PM-1), or a second precursor component having the following formula (F-PM-2), or a mixture or combination of both the first precursor component and the second precursor component:

M^{b}_{q-p}(M^{a}L_{q})ₘ (F-PM-1)

M^{b}Lⱼ (F-PM-2)

where j is an integer or non-integer, and m−1≤j≤m.

In particularly advantageous embodiments, the first compound and the second compound are water soluble at a temperature in the range from 20 to 80° C., preferably water soluble at room temperature. In these embodiments, desirably M^{d} and M^{e} independently provide an alkali metal ion, a proton, or an ammonium group in (F-I-1) and (F-I-2), respectively. It is possible to mix the first material and/or the second materials in solid form to produce the first solid precursor without the need of the use of a solvent. Desirably, the first compound and/or the second compound are dispersed (e.g., dissolved) in a solvent such as water to form a dispersion (e.g., a solution, a suspension, and/or a colloidal system) of compounds (F-I-1) and/or (F-I-2), which is then allowed to contact and react with the second material having a formula (F-II). Water is a preferred solvent over other solvents due to its environmental friendliness and low safety risk. Such dispersion of the first compound and/or the second compound may take the form of a gel distributed in a solvent.

In the second material having formula (F-II), A can be an anion such as a NO₃⁻, a halogen anion, a CH₃COO⁻, a citric anion, and the like. Desirably, the second material having a formula (F-II) can be a water soluble compound such as a salt of metal M^{b}, e.g., a nitrate, a nitrite, a fluoride, a chloride, a bromide, a acetate, a citrate, and the like. Desirably, a liquid dispersion (such as an aqueous solution, an aqueous suspension, or an aqueous colloidal system) of a first material is combined with a liquid dispersion (such as an aqueous solution, an aqueous suspension, or an aqueous colloidal system) of a second material to produce a first solid precursor precipitating from the liquid phase and separable from the liquid phase. Alternatively, a liquid dispersion of a first material can be combined with a solid of a second material, mixed, and allowed to react to produce a first solid precursor, and vice versa.

The first precursor can comprise a solid of a compound represented by formula (F-PM-1), and/or a solid of a compound represented by formula (F-PM-2), and/or a solid of a mixture of a material represented by formula (F-PM-1) and a material represented by formula (F-PM-2).

The first precursor can comprise an ionic network having a formula (F-PM-1), and/or an ionic network having a formula (F-PM-2), substantially the same or similar to those as described above in association with the precursor material as an aspect of this disclosure. Such ionic network may present it itself as a gel dispersed in a solvent such as water.

The first precursor can comprise an interconnected ionic network having a mixture and/or combination of portions which collectively can be represented jointly by a formula (F-PM-1) and a formula (F-PM-2), substantially the same or similar to those as described above in association with the precursor material as an aspect of this disclosure.

The processes for making a catalyst precursor can further comprise a step (iv) of adding a third material comprising a metal element M^{c}, to the first solid precursor to obtain a second solid precursor. It is highly desirable that the third material is a water soluble compound of M^{c}. For example, the third material can be a nitrate, a nitrite, a chloride, a fluoride, a bromide, an acetate, a citrate, and the like, of metal M^{c}, or a mixture or combination thereof.

Step (iv) can be effected at least partly simultaneously in step (iii), wherein the first material, the second material and the third material are combined, and after step (iii), the first solid precursor is separated from a liquid phase in the liquid dispersion, and the first solid precursor carries a quantity of the third material. Additionally or alternatively, step (iv) can be effected at least partly after step (iii), and the process further comprises: (iiia) after step (iii), separating the first solid precursor from a liquid phase in the liquid dispersion; (iiib) optionally washing the separated first solid precursor using a solvent; and subsequently (iiic) impregnating the separated first solid precursor with a dispersion of the third material in a liquid. In more specific embodiments, the process can further comprise, after step (iiic), drying and/or calcining the impregnated first solid precursor to obtain the second solid precursor. Preferred liquid dispersions are aqueous dispersions comprising water as a solvent, more preferably as a sole solvent.

Owing to the unique processes for making the catalyst precursor, metals M^{a}, and/or M^{b} and/or M^{c} can be distributed substantially homogeneously in the catalytic precursor. So can the carbon and nitrogen atoms. The atoms of metals M^{a} and/or M^{b} can be directly bonded to the carbon and/or nitrogen atoms in the ligands CN⁻, SCN⁻, and/or OCN⁻. The homogeneous distribution of the metal atoms in the catalyst precursor enables the homogeneous distribution thereof in the catalytic component made from them via thermal decomposition, as described below.

It is highly advantageous that the metal carbide(s) and/or the metal nitride(s) are highly dispersed in the catalytic component. The metal carbide(s) and/or the metal nitride(s) can be substantially homogeneously distributed in the catalytic component, resulting in a highly dispersed distribution, which can contribute to a high catalytic activity of the catalytic component.

### Producing Catalytic Components and Catalyst Compositions

To obtain a catalytic component, one can further perform a step (v) to a catalyst precursor made pursuant to the description above:

(v) heating the first solid precursor and/or the second solid precursor at a temperature equal to or greater than 200° C., preferably in a range from 200 to 800° C., in the presence of an inert atmosphere for a period of at least 1 minute to obtain a catalytic component.

The inert atmosphere protecting the first solid precursor and/or the second solid precursor and the catalytic component after completion of thermal decomposition is absent of a gas that can oxidize the first solid precursor and/or the second solid precursor such as oxygen. It may be desirable that the inert atmosphere is absent of a gas that can reduce the first solid precursor and/or the second solid precursor such as hydrogen. It may be desirable that the inert atmosphere is a flowing stream of gas having a low water partial pressure. Thus, the inert atmosphere may comprise nitrogen gas, argon, helium, neon, mixtures of two or more thereof, and the like.

The first precursor and/or the second precursor material are then heated to an elevated temperature from T1 to T2° C., where T1 and T2 can be, independently, e.g., 200, 250, 300, 350, 400, 450, 500, 550, 600, 700, 750, 800, 850, 900, 950, or 1000, as long as T1<T2. Preferably T1=200, and T2=800. More preferably T1=200, and T2=600. Still more preferably T1=300, and T2=500. At such elevated temperature, the first precursor and/or the second precursor are heated for a period of at least 1 minutes under the protection of the inert atmosphere. Preferably, the heating period can range from t1 to t2 hours, where t1 and t2 can be, independently, e.g., 0.1, 0.2, 0.3, 0.4, 0.5, 0.6, 0.7, 0.8, 0.9, 1, 2, 4, 5, 6, 8, 10, 12, 14, 15, 16, 18, 20, 22, 24, 25, 26, 28, 30, 32, 34, 35, 36, 38, 40, 42, 44, 45, 46, or 48, as long as t1<t2.

The exposure of the first precursor and/or the second precursor to the elevated thermal decomposition temperature for the heating period results in thermal decomposition thereof to obtain a catalytic component. Upon completion of the heating period, the catalytic component may be used as is as a catalyst composition in a conversion reactor, or cooled down under the protection of the inert atmosphere, after which it can be combined with other components of the catalyst composition, such as a binder, a support, a co-catalyst, and the like, to make a catalyst composition.

The thus prepared catalytic component in step (v) can desirably comprise M^{a}, M^{b}, optionally M^{c}, carbon, nitrogen, and optionally sulfur, at a molar ratio of M^{b}, M^{c}, carbon, nitrogen, and sulfur to M^{a} of r1, r2, r3, r4, and r5, respectively, indicated below:

M^{b}:M^{c}:C:N:S:M^{a}=r1:r2:r3:r4:r5:1, where:

0.1≤r1≤1.5;

0≤r2≤0.5;

0<r3≤1;

0<r4≤1; and

0≤r5≤1,

where M^{a} is selected from manganese, iron, cobalt, and combinations and mixtures of two or more thereof at any proportion, M^{b} is selected from aluminum, gallium, indium, thallium, chromium, scandium, yttrium, the lanthanide series, the actinide series, and any combination or mixture of two or more thereof at any proportion, M^{c} is selected from alkali metals, copper, silver, and any combinations or mixtures of two or more thereof at any proportion.

It may be desirable that at least a portion of the carbon in the catalytic component is present as a metal carbide of one or more of M^{a}, M^{b}, and M^{c}, and/or at least a portion of the nitrogen in the catalytic component is present as a metal nitride of one or more of M^{a}, M^{b}, and M^{c}, as determined by XRD of the catalytic component. More desirably, at least a portion of the carbon in the catalytic component is present as a metal carbide of one or more of M^{a} and M^{b}, and/or at least a portion of the nitrogen in the catalytic component is present as a metal nitride of one of more of M^{a} and M^{b}, as determined by XRD of the catalytic component. Without intending to be bound by a particular theory, it is believed the presence of the metal carbide(s) and the metal nitride(s) benefits at least in part to the catalytic effect of the catalytic component in the catalyst composition of this disclosure.

By using the process for making the catalyst precursor and the process for making the catalyst composition, one can surprisingly make a catalyst composition comprising metal carbide(s), metal nitride(s), or combination of both, at a low temperature of no higher than 800° C., by thermal decomposition of the first solid precursor and/or the second solid precursor comprising both carbon and nitrogen in the form of CN⁻, OCN⁻, and/or SCN⁻. Without intending to be bound by a particular theory, such low processing temperature is enabled by the presence of the metal(s), carbon, and nitrogen atoms in close proximity to each other in the structure of the first solid precursor and the second solid precursor.

Due to the substantially homogeneous distribution of the metal atoms M^{a} and M^{b} in the catalyst precursor, the directly bonding between the metal atoms to the carbon and/or nitrogen atoms in the ligands in the catalyst precursor, and the substantially homogeneous distribution of carbon and nitrogen atoms in the catalyst precursor, metal carbides and/or metal nitride phases of M^{a} and/or M^{b} can be formed in the thermal decomposition process to make the catalytic component, where the metals M^{a} and/or M^{b}, and the carbide/nitride phases thereof, can be substantially homogeneously distributed in the catalyst component thus made. Homogeneous distribution of metal(s) results in highly dispersed distribution thereof, large number of catalytically effective sites on the catalytic component, and high catalyst activity of the catalytic component.

The thus made catalytic component can be used as is as a catalyst composition for its intended use (e.g., converting syngas), i.e., as a bulk catalyst. The freshly thermally decomposed catalytic component may undergo chemical and/or physical changes when in contact with ambient air, including oxidation, water absorption, and the like. Therefore, it is highly desirable to conduct the thermal decomposition step (v) in a reactor the catalyst composition is intended for, such as a syngas converting reactor. Following the thermal decomposition, a feed (e.g., a feed comprising syngas) can replace the inert atmosphere used in step (v), whereupon a chemical process (e.g., a syngas conversion process) can be initiated in the presence of the catalyst composition under desirable conversion conditions.

Alternatively, after step (v), one can combine the catalytic component with a catalyst support material, a co-catalyst, or a solid diluent material, to form a catalyst composition. Suitable catalyst support materials for combining with the catalytic component were described earlier in this disclosure in connection with the catalyst composition. The combination of the support material and the catalytic component can be processed in any known catalyst forming processes, including but not limited to grinding, milling, sifting, washing, drying, calcination, and the like, to obtain a catalyst composition. The catalyst composition may be then disposed in an intended reactor to perform its intended function, such as a syngas converting reactor in a syngas converting process.

It is contemplated that prior to step (v), the first solid precursor and/or the second solid precursor may be combined with a catalyst support material to obtain a mixture thereof, which is subsequently subject to step (v). In such processes, the first solid precursor and/or the second solid precursor are desirably disposed on the internal and/or external surfaces of the support material. In the subsequent step (v), the catalyst precursor(s) thermally decompose to leave a catalytic component on the surface of the support material, to form a catalyst composition. Likewise, the subsequent step (v) can be desirably performed in a reactor where the catalyst composition is normally used, such as a syngas converting reactor. Alternatively, step (v) can be performed in a reactor other than the reactor the catalyst composition is intended for to obtain a catalyst composition comprising a support material and the catalytic component, which can be stored, shipped, and then disposed in a reactor it is intended for.

It is also contemplated that prior to step (v), the first solid precursor and/or the second solid precursor may be combined or formed with a precursor of a support material to obtain a support/catalytic component precursor mixture. Suitable precursors of various support materials can include, e.g., alkali metal aluminates, water glass, a mixture of alkali metal aluminates and water glass, a mixture of sources of a di-, tri-, and/or tetravalent metal, such as a mixture of water-soluble salts of magnesium, aluminum, and/or silicon, chlorohydrol, aluminum sulfate, or mixtures thereof. The support/catalytic component precursor mixture is subsequently subject to step (v) together, resulting in the formation of the catalytic component and the support material substantially in the same step. Likewise, the subsequent step (v) can be desirably performed in a reactor where the catalyst composition is normally used, such as a syngas converting reactor. Alternatively, step (v) can be performed in a reactor other than the reactor the catalyst composition is intended for to obtain a catalyst composition comprising a support material and the catalytic component, which can be stored, shipped, and then disposed in a reactor it is intended for.

### Processes for Converting Syngas

The catalyst composition of this disclosure can be used in any process where the relevant metal(s) and/or the metal carbide(s) and/or the metal nitride(s) can perform a catalytic function. The catalyst composition of this disclosure can be particularly advantagedly used in processes for converting syngas into various products such as alcohols and olefins, particularly C1-C5 alcohols, such as C1-C4 alcohols, and C2-C5 olefins (particularly C2-C4 olefins), such as the Fischer-Tropsch processes. The Fischer-Tropsch process is a collection of chemical reactions that converts a mixture of carbon monoxide and hydrogen into hydrocarbons and/or alcohols. These reactions occur in the presence of metal catalysts, typically at temperatures of 100-500° C. (212-932° F.) and pressures of one to several tens of atmospheres.

The term “syngas” as used herein relates to a gaseous mixture consisting essentially of hydrogen (H₂) and carbon monoxide (CO). The syngas, which is used as a feed stream, may include up to 10 mol % of other components such as CO₂ and lower hydrocarbons (lower HC), depending on the source and the intended conversion processes. Said other components may be side-products or unconverted products obtained in the process used for producing the syngas. The syngas may contain such a low amount of molecular oxygen (O₂) so that the quantity of O₂ present does not interfere with the Fischer-Tropsch synthesis reactions and/or other conversion reactions. For example, the syngas may include not more than 1 mol % O₂, not more than 0.5 mol % O₂, or not more than 0.4 mol % O₂. The syngas may have a hydrogen (H₂) to carbon monoxide (CO) molar ratio of from 1:3 to 3:1. The partial pressures of H₂ and CO may be adjusted by introduction of inert gas to the reaction mixture.

Syngas can be formed by reacting steam and/or oxygen with a carbonaceous material, for example, natural gas, coal, biomass, or a hydrocarbon feedstock through a reforming process in a syngas reformer. The reforming process can be based on any suitable reforming process, such as Steam Methane Reforming, Auto Thermal Reforming, or Partial Oxidation, Adiabatic Pre Reforming, or Gas Heated Reforming, or a combination thereof. Example steam and oxygen reforming processes are detailed in U.S. Pat. No. 7,485,767.

The syngas formed from steam or oxygen reforming includes hydrogen and one or more carbon oxides (CO and CO₂). The hydrogen to carbon oxide ratio of the syngas produced will vary depending on the reforming conditions used. The syngas reformer product(s) should contain H₂, CO and CO₂ in amounts and ratios which render the resulting syngas blend suitable for subsequent processing into either oxygenates comprising methanol/dimethyl ether or in Fischer-Tropsch synthesis.

The syngas from reforming to be used in Fischer-Tropsch synthesis may have a molar ratio of H₂ to CO, unrelated to the quantity of CO₂, of 1.9 or greater, such as from 2.0 to 2.8, or from 2.1 to 2.6. On a water-free basis, the CO₂ content of the syngas may be 10 mol % or less, such as 5.5 mol % or less, or from 2 mol % to 5 mol %, or from 2.5 mol % to 4.5 mol %.

It is possible to alter the ratio of components within the syngas and the absolute CO₂ content of the syngas by removing, and optionally recycling, some of the CO₂ from the syngas produced in one or more reforming processes. Several commercial technologies are available (e.g. acid gas removal towers) to recover and recycle CO₂ from syngas as produced in the reforming process. In at least one embodiment, CO₂ can be recovered from the syngas effluent from a steam reforming unit, and the recovered CO₂ can be recycled to a syngas reformer.

Suitable Fischer-Tropsch catalysis procedures may be found in: U.S. Pat. Nos. 7,485,767; 6,211,255; and 6,476,085; the relevant portions of their contents being incorporated herein by reference. The catalyst composition may be contained in a fixed bed reactor, a fluidized bed reactor, or any other suitable reactor. The reaction conditions may include contacting the catalyst composition with syngas, to provide a reaction mixture, at a pressure of 1 bar to 50 bar, at a temperature of 150° C. to 450° C., and/or a gas hourly space velocity of 1000 h⁻¹ to 10,000 h⁻¹ for a reaction period.

The reaction conditions may include a wide range of temperatures. In at least one embodiment, the reaction temperature ranges from 100° C. to 450° C., such as from 150° C. to 350° C., such as from 200° C. to 300° C. For certain catalyst compositions, lower temperature ranges might be preferred, but with a catalyst composition including cobalt metal, higher temperatures are tolerated. For example, a catalyst composition including cobalt metal may be mixed at reaction temperatures of 250° C. or greater, such as from 250° C. to 350° C., or from 250° C. to 300° C.

The reaction conditions may include a wide range of pressures. In at least one embodiment, the absolute reaction pressure ranges from p1 to p2 kilopascal (“kPa”), wherein p1 and p2 can be, independently, e.g., 100, 150, 200, 250, 300, 350, 400, 450, 500, 600, 700, 800, 900, 1000, 1500, 2000, 2500, 3000, 3500, 4000, 4500, or 5,000, as long as p1<p2.

Gas hourly space velocities used for converting the syngas to olefins and/or alcohols can vary depending upon the type of reactor that is used. In one embodiment, gas hourly space velocity of the flow of gas through the catalyst bed is from 100 hr⁻¹ to 50,000 hr⁻¹, such as from 500 hr⁻¹ to 25,000 hr⁻¹, from 1000 hr⁻¹ to 20,000 hr⁻¹, or from 100 hr⁻¹ to 10,000 hr⁻¹.

Reaction conditions may have an effect on the catalyst performance. For example, selectivity on a carbon basis is a function of the probability of chain growth. Factors affecting chain growth include the temperature of the reaction, the gas composition and the partial pressures of the various gases in contact with the catalyst composition. Altering these factors may lead to a high degree of flexibility in obtaining a type of product in a certain carbon range. Without being limited by theory, an increase in operating temperature shifts the selectivity to lower carbon number products. Desorption of growing surface species is one of the main chain termination steps and since desorption is an endothermic process so a higher temperature should increase the rate of desorption which will result in a shift to lower molecular mass products. Similarly, the higher the CO partial pressure, the more catalyst surface that is covered by adsorbed monomers. The lower the coverage by partially hydrogenated CO monomers, the higher the probability of chain growth. Accordingly, it is probable that the two key steps leading to chain termination are desorption of the chains yielding alkenes and hydrogenation of the chains to yield alkanes.

### EXAMPLES

### Part A. Catalyst Synthesis and XRD Characterization

### Example A1

### Preparation of La(III)[Co(III)(CN)₆] and Similar Bimetallic Catalyst Precursors

In a 500 cc flask containing 200 cc water, potassium hexacyanocobaltate(III) was dissolved (100 mmol, 33.4 g). To this solution, a trivalent metal nitrate (e.g., 100 mmol, 43.3 g La(NO₃)₃.6H₂O) was added. The initial clear solution turned into a viscous slurry upon stirring at 25-50° C. for 2-10 hrs. The reaction product was filtered and washed using deionized water to yield of ˜35 g solid. The solid was dried and used as a catalyst precursor. The reaction can be illustrated below:

La(NO₃)₃+K₃[Co(III)(CN)₆]→La[Co(III)(CN)₆↓+3 KNO₃

The solid catalyst precursor was then thermally decomposed under of a flowing stream of nitrogen at a temperature between 300 and 500° C. to obtain a catalyst component. The thus made catalytic component can be analyzed by XRD to identify the respective phases.

Similarly, lanthanide cyanoferrate can be made:

La(NO₃)₃+K₃[Fe(III)(CN)₆]→La[Fe(III)(CN)₆↓+3 KNO₃

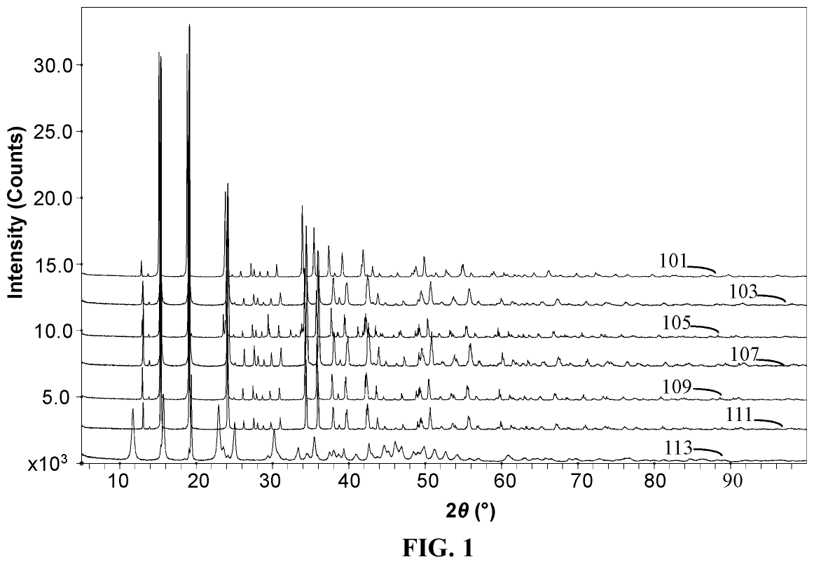

Multiple catalytic components comprising two metal elements—iron and a lanthanide—were made according to the procedure of this Example A1. The XRD analysis results of the catalyst precursors after drying are included in FIG. 1, and their solid precursor compositions are listed in TABLE I below:

TABLE I

| Reference No. | Solid Catalyst Precursor |
|---|---|
| 101 | Nd(III)[Fe(III)(CN)₆] |
| 103 | Ho(III)[Fe(III)(CN)₆] |
| 105 | Gd(III)[Fe(III)(CN)₆] |
| 107 | Er(III)[Fe(III)(CN)₆] |
| 109 | Tb(III)[Fe(III)(CN)₆] |
| 111 | Dy(III)[Fe(III)(CN)₆] |
| 113 | Y(III)[Fe(III)(CN)₆] |

### Example A2

### Preparation of Co—La—Mn and Similar Trimetallic Catalyst Precursors

In a 500 cc flask containing 200 cc water, potassium hexacyanocobaltate(III) was dissolved (100 mmol, 33.4 g). To this solution, 8.5 g (56 mmol) of manganese sulfate was added followed by addition of a trivalent metal nitrate (e.g., 100 mmol, 43.3 g La(NO₃)₃.6H₂O) and 3.8 g (50 mmol) of NH₄SCN. The initial clear solution turned into a viscous slurry upon stirring at 25-50° C. for 2-10hrs. The reaction product was filtered to yield of ˜45 g solid. The solid was water washed, dried and then used as catalyst precursor. The solid is believed to be a mixture comprising the following, where L, the same or different at each occurrence, is CN⁻ or SCN⁻, and x can be, independently, any integer or non-integer number ranging from 2 to 6: La[CoLₓ]; La[MnLₓ]; Mn[CoLₓ]; Co[MnLₓ]; La(SCN)₃; Mn(SCN)₂; and La(SCN)₃.

The solid catalyst precursor may be an ionic network wherein some of the CN⁻ and SCN⁻ bi-dentate ligand complex with two metal ions. Where there are defects in the network, some NH₄⁺ or K⁺ ions may be present.

The water soluble products in the above reaction can include: K₂SO₄; (NH₄)₂SO₄; KNH₄SO₄; KCN; KSCN; NH₄CN; and NH₄SCN, which were removed from the solid catalyst precursor by water washing.

The dried solid catalyst precursor was used in Example B1 below in an exemplary syngas conversion process, where it was studied for its catalytic effect.

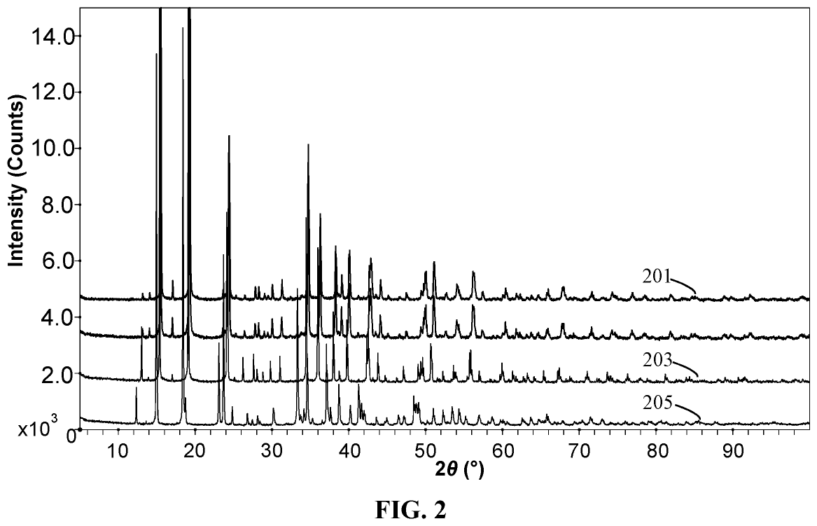

Multiple trimetallic catalytic components of this disclosure were made using the same procedure of this Example A2 by replacing La(NO₃)₃ with another lanthanide nitrate. XRD diagrams of several catalyst precursors thus made are included in FIG. 2, and elements believed to be present in the respective solid catalyst precursors are listed in TABLE II below:

TABLE II

| Reference No. | Elements in Solid Catalyst Precursor |
|---|---|
| 201 | Y, Co, Mn, C, S |
| 203 | Gd, Co, Mn, C, S |
| 205 | La, Co, Mn, C, S |

The dried solid catalyst precursor was then thermally decomposed at a temperature around 450° C., to obtain a catalytic component. XRD of the catalytic component showed cobalt 17.08 wt %, manganese 9.193 wt %, lanthanum 33.89 wt %, and sulfur 4.928 wt %, based on the total weight of the catalytic component.

Another catalytic component made by the procedure of this Example A2 comprising cobalt, manganese, and yttrium was found to comprise yttrium 28.5 wt %, cobalt 23.7 wt %, manganese 13 wt %, and Sulfur 2.2 wt %, based on the total weight of the catalytic component, according to the XRD diagram. The solid catalyst precursor to this catalytic component was used in Example B2 below in an exemplary syngas conversion process, where it was studied for its catalytic effect.

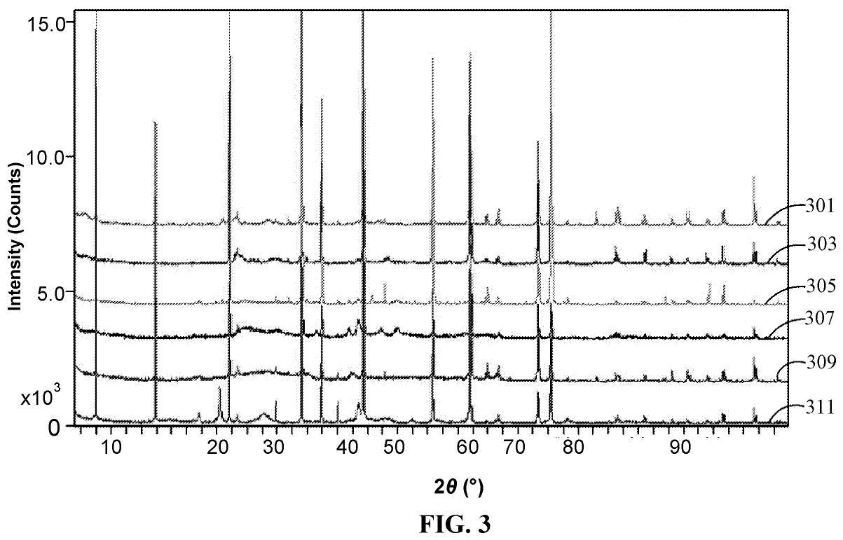

A series of bimetallic or trimetallic catalyst components of this disclosure were made using the procedures of Example A1 or A2, and tested in syngas conversion processes. Upon testing, some of the used catalytic components were then evaluated by XRD. XRD patterns of six of them are presented in FIG. 3. Metals contained in these catalytic components are listed in TABLE III below:

TABLE III

| Reference No. | Metal Elements in Catalytic Component |
|---|---|
| 301 | Pr, Co, Cu |
| 303 | Gd, Co, Cu |
| 305 | Y, Co, Mn |
| 307 | Y, Co, Cu |
| 309 | Er, Fe |
| 311 | La, Co |

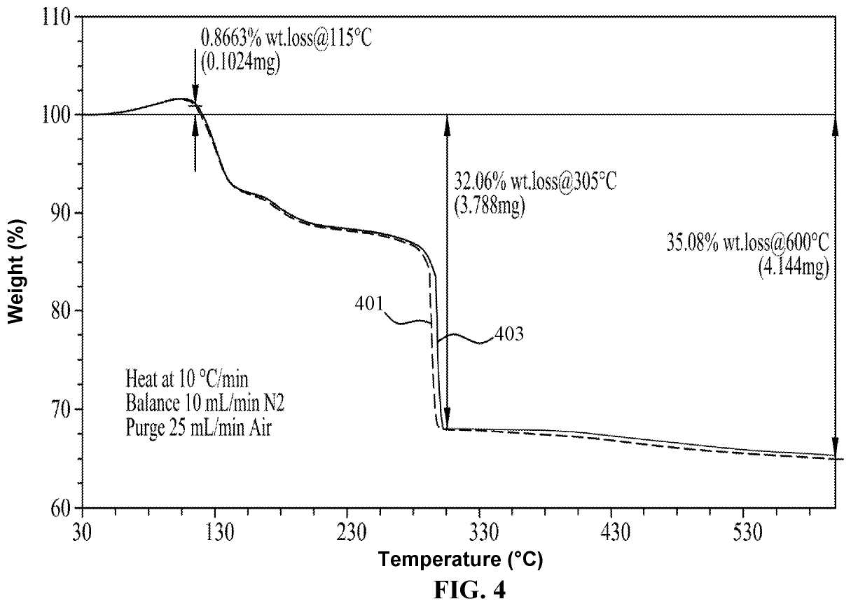

Thermogravimetric analysis of a catalyst precursor of an embodiment of this disclosure comprising holmium hexacyanoferrate (Ho(III)[Fe(III)(CN)₆]) was conducted twice at a temperature elevation rate of 10° C./min under air purge. The analysis results are shown in FIG. 4 as weight-temperature curves. The dotted curve 401 shows analysis result of a first sample taken from the catalyst precursor. The solid curve 403 shows analysis result of a second sample taken from the same catalyst precursor, but analyzed the day the first sample was analyzed. As can be seen from FIG. 4, the two curves align with each other very closely, indicating the precursor material did not undergo significant change overnight. The curves clearly show that around 305° C., significant change occurred to the precursor material resulting in total weight loss of 32.06%, indicating thermal decomposition of the precursor.

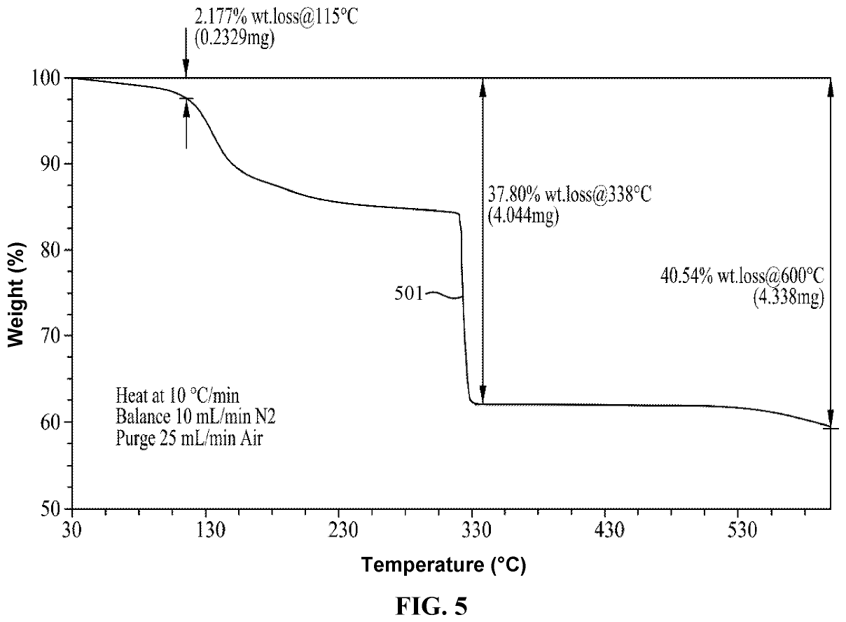

Similarly, FIG. 5 shows a thermogravimetric analysis result of a catalyst precursor 501 of another embodiment of this disclosure comprising gadolinium hexacyanocobaltate (Gd(III)(Co(III)(CN)₆) at a temperature elevation rate of 10° C./min under air purge. Around 338° C., significant changes occurred to the catalyst precursor, resulting in a total weight loss of 37.80%, indicating thermal decomposition of the catalyst precursor.

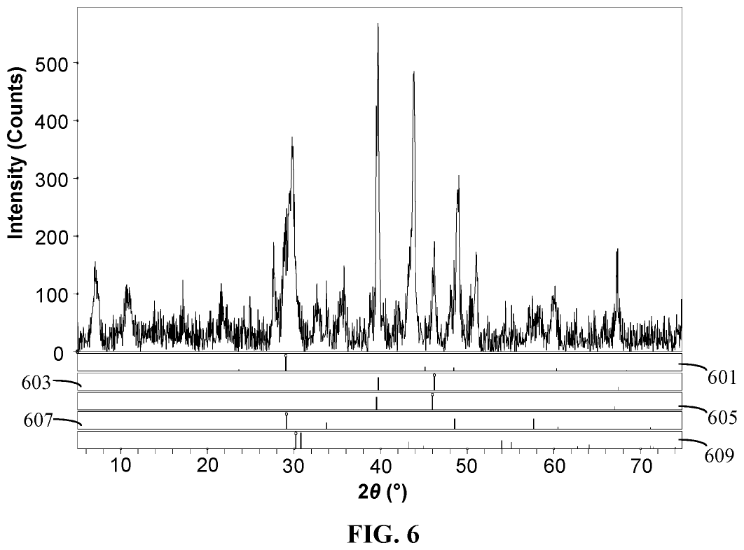

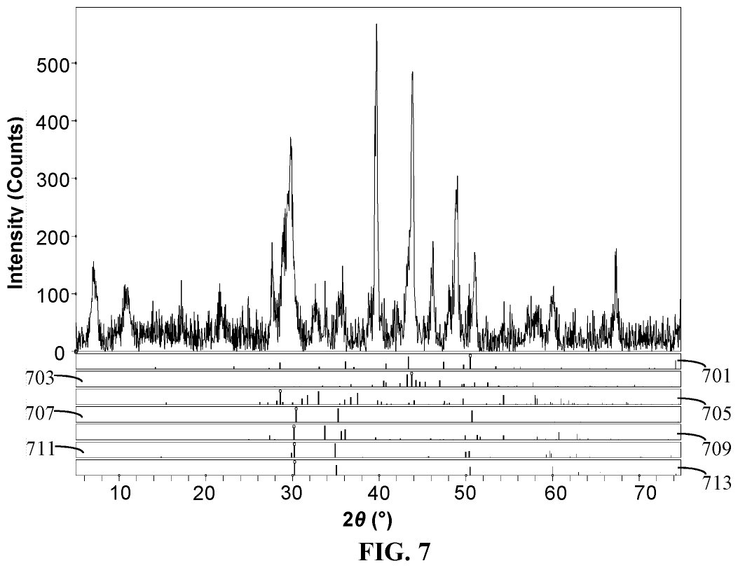

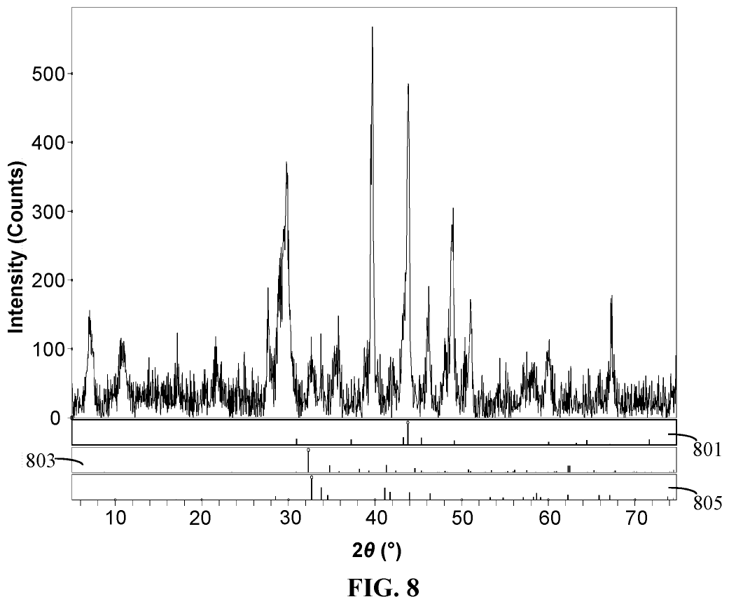

A sample of a catalyst component comprising cobalt, manganese, and yttrium prepared pursuant to the procedure of this Example A2 was characterized by powder XRD. The XRD diagram and the various peak groups identified by reference numerals are provided in FIGS. 6, 7, and 8. These peak groups match with known peaks of various phases pursuant to International Center for Diffraction Data (“ICDD”) XRD peak library as listed in TABLE IV below. The peak groups for carbon (01-079-1473), carbon nitride (01-078-1747, C₃N₄), yttrium (01-089-9233), and yttrium manganese (Mn₂Y, 03-066-0003), identifiable from the XRD graph, are not provided in FIGS. 6, 7, and 8.

Clearly in the catalytic component, multiple phases of carbides of several metals and multiple phases of nitrides of several metals are present as evidenced by the XRD diagram. Without intending to be bound by a particular theory, it is believed that the presence of these carbides and/or nitride phases are conducive to the catalytic activity of the catalytic component, especially for the purpose of converting syngas.

In the catalytic component, metal and metal alloy phases are also present. These phases may perform catalytic effect as well.

TABLE IV

| Peak Group | ICDD Library | | Phase |
|---|---|---|---|
| in XRD | Identification No. | Phase Name | Composition |
| — | 01-079-1473 | Carbon | C |
| — | 01-078-1747 | Carbon Nitride | C₃N₄ |
| — | 01-089-2933 | Yttrium | Y |
| — | 03-066-0003 | Yttrium Manganese | Mn₂Y |
| 601 | 01-078-1747 | Carbon Nitride | C₃N₂ |
| 603 | 01-083-8039 | Cobalt Nitride | CoN |
| 605 | 01-083-8037 | Manganese Nitride | MnN |
| 607 | 01-080-5705 | Yttrium Nitride | YN |
| 609 | 01-081-0300 | Manganese Nitride | Mn₃N₂ |
| 701 | 01-081-9789 | Manganese Carbide | Mn₁₁C₂ |
| 703 | 01-080-1700 | Manganese Carbide | Mn₅C₂ |
| 705 | 01-088-0569 | Yttrium Carbide | Y₄(C₂)₂C |
| 707 | 01-080-5687 | Yttrium Carbide | YC |
| 709 | 01-081-4226 | Yttrium Carbide | Y₁₅C₁₉ |
| 711 | 01-072-6343 | Yttrium Carbide | Y₂C |
| 713 | 01-073-0501 | Yttrium Carbide | YC_{0.40} |
| 801 | 03-065-8990 | Cobalt Yttrium | Co₅Y |
| 803 | 03-065-5573 | Cobalt Yttrium | Co₇Y₉ |
| 805 | 01-073-0117 | Yttrium Cobalt | YCo |

It is totally surprising that carbide phases and nitride phases were formed in the catalytic component at such low thermal decomposition temperature employed. In typical processes for making metal carbides involving the reaction of a metal source and a carbon source, one would have to run the reaction at above 800° C. to effect the formation of a metal carbide. In typical processes for making metal nitrides involving the reaction of a metal source and ammonia, the temperature required is higher than 650° C. Yet, by utilizing the processes of this disclosure involving the formation of a catalyst precursor comprising the metals, carbon, and nitrogen in the compounds and/or ionic network, one can obtain a catalyst component comprising both a metal carbide phase and a metal nitride phase at a temperature significantly lower than 800° C., such as lower than 600° C., and even lower than 500° C. Such low thermal decomposition temperature enables the in-situ thermal decomposition of the catalyst precursor to produce a catalytic component in a conversion reactor that the catalytic component is intended for, which is particularly advantageous if the conversion process is typically conducted around or lower than the thermal decomposition. Further, the metal carbide phase(s) and the metal nitride phase(s) are believed to be distributed intimately and substantially homogenously with other phases, including the individual metal phases, the mixed metal phases, the carbon phase, and the carbon nitride phases in the catalytic component, providing highly dispersed distribution thereof in the catalytic component, resulting in high catalytic activity as demonstrated by the syngas conversion process examples below. This is contrary to conventional processes for making metal carbide(s) and metal nitride(s), which tend to result in in-homogenous distribution thereof, typically more preferentially on the surface only, resulting in likely low dispersion and low catalytic activity.

### Comparative Example A3

### Preparation of a Trimetallic Oxide Catalyst

A La—Co—Mn oxide system, compositionally similar to the catalytic component in Example B1 above in terms of La, Co, and Mn content, synthesized via aqueous phase co-precipitation of La(NO₃)₃, Co(NO₃)₂ and Mn(NO₃)₂ with Na₂CO₃ and then calcined in air at between 450-550° C. The thus made comparative catalyst composition comprises oxides and mixed oxides of La, Co, and Mn, and is believed to be free of metal carbide and metal nitride phases.

### Part B: Processes for Converting Syngas

### Example B1

Process for Converting Syngas Using a Co—La—Mn Trimetallic Catalytic Component of this Disclosure

A 3:2 mixture (by weight) of the Co—La—Mn trimetallic catalyst precursor (60 wt %) made in the preceding Example A2 and silicon carbide (40 wt %) (both components sized between 40-60 mesh) were loaded into a fixed-bed reactor system. The catalyst was dried at 110° C. under an N₂ purge at 15 bar absolute pressure and GHSV=2000 h⁻¹ for 2 h. Maintaining the N₂ purge, reactor pressure, and GHSV, the reactor temperature was then increased to 400° C. at a 1° C./min ramp rate. The reactor was kept at 400° C. for 2 h, whereupon the catalyst precursor is believed to have thermally decomposed to form a Co—La—Mn/C/N trimetallic catalytic component comprising metal carbide and/or metal nitride phases. The mixture of the in-situ converted catalytic component and the silicon carbide diluent can be regarded as catalyst composition of this disclosure. The reactor was then cooled to the Fischer-Tropsch synthesis temperature (e.g., 250° C.). Once the temperature stabilized, the reactor feed was switched to a mixture of H₂ and CO (e.g., syngas). The range of conditions explored were as follows: (1) temperature, 150-350° C.; (2) pressure, 1-50 bar; (3) H₂:CO ratio, 1:3 to 3:1, and (4) GHSV, 1000-10,000 h⁻¹.

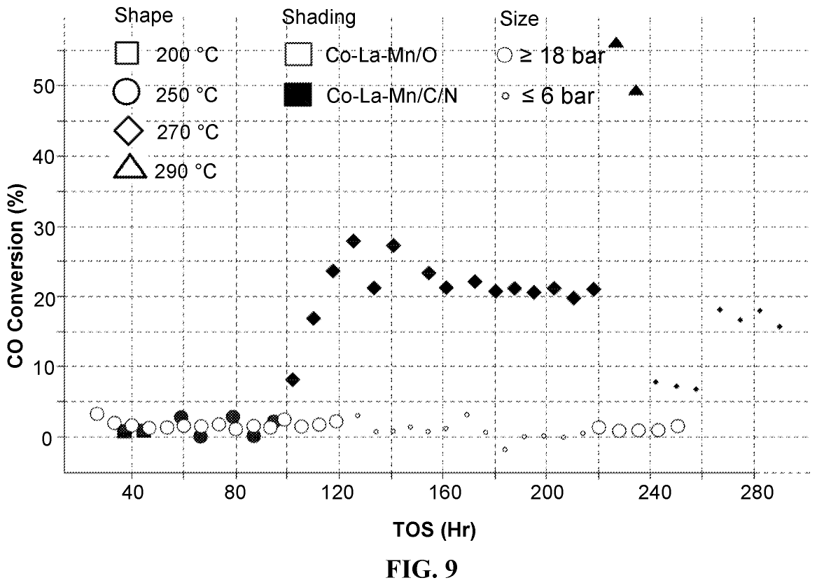

FIG. 9 shows CO conversion as a function of time one stream of this catalyst composition (the non-shaded data points). Further discussion of this figure is provided below in connection with comparative Example B3 below.

### Example B2

Process for Converting Syngas Using a Co—Y—Mn Trimetallic Catalytic Component of this Disclosure

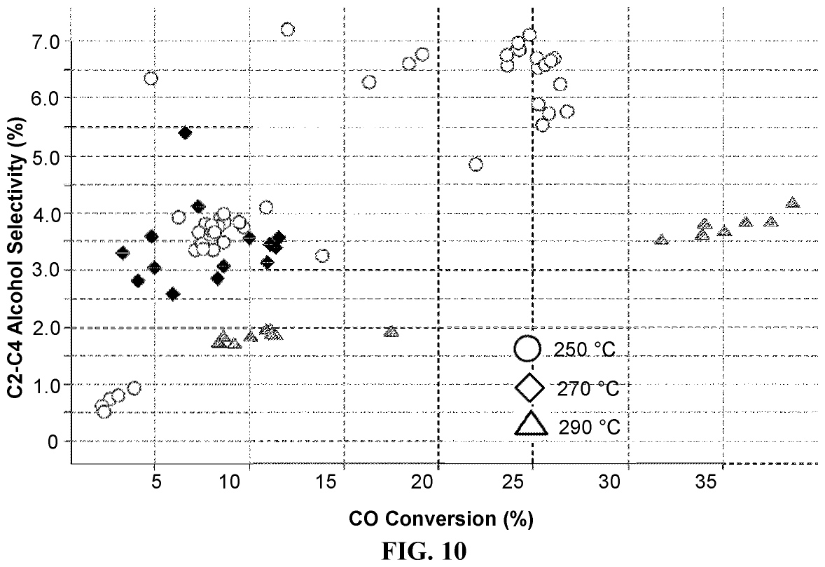

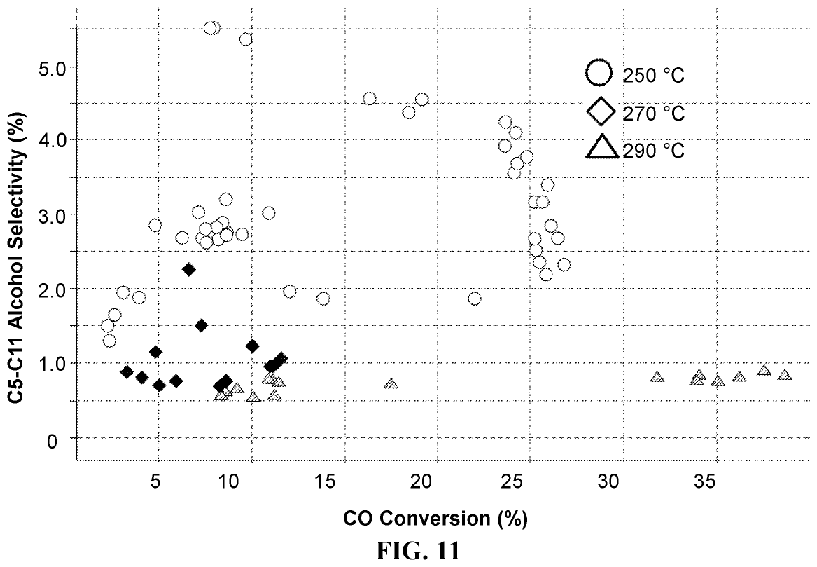

Following procedure identical to that in Example B1 above, the trimetallic Co—Y—Mn catalyst precursor made in the preceding Example A2 was mixed with silicon carbide diluent, and then converted into a catalytic component in a syngas reactor at an elevated temperature, whereupon it thermally decomposed to form a Co—Y—Mn/C/N trimetallic catalytic component, which was then subsequently tested for syngas conversion. The in-situ made catalytic component is believed to comprise metal carbide and/or metal nitride phases. The C2-C4 alcohol selectivity as a function of CO conversion in this syngas conversion process in the presence of this catalyst composition is depicted in FIG. 10. The C5-C11 alcohol selectivity as a function of CO conversion of the syngas conversion process is depicted in FIG. 11. In these two figures, the different shapes of the data points denote different conversion temperatures: circle (shaded or non-shaded) for 250° C., rhombus (shaded or non-shaded) for 270° C., and triangle (shaded or non-shaded) for 290° C., as shown in the legends in the figure. In FIG. 10, C2-C4 alcohol selectivity as high as 7% were observed at 250° C., and this selectivity goes down at higher temperatures, favoring olefin formation. At each reaction temperature, the alcohol selectivity does not change appreciably with increase in conversion.

### Example B3

### Comparative Example

### Process for Converting Syngas Using a Co—La—Mn Trimetallic Oxide Catalyst Composition

In this comparative example, the catalyst composition prepared in the preceding Example A3, a Co—La—Mn trimetallic oxide-based catalyst composition, which is believed to be substantially free of a metal carbide and/or a metal nitride phase, was tested in a syngas conversion reactor. Test data of this comparative catalyst composition, and those of the inventive Co—La—Mn/C/N catalyst composition in Example B1 above, are presented in FIG. 9. In this figure, the shapes of the data points denote reaction temperature: square for 200° C., circle for 250° C., rhombus for 270° C., and triangle for 290° C.; the shadings of the data points denote the catalyst: shaded for the inventive Co—La—Mn/C/N catalyst composition of Example B 1, and non-shaded for the comparative Co—La—Mn/O catalyst; and the sizes of the data points denote reaction pressure: large for an absolute pressure of at least 1800 kPa, and small for at most 600 kPa. As can be seen, the comparative catalyst, even though it comprises Co, La, and Mn at comparable amounts to the catalytic component of Example B1, demonstrated very little catalytic activity: at T=250-290° C., 2:1 H₂/CO ratio, 18 bar, and GHSV=2000 h⁻¹, less than 3% CO conversion was observed with methane as the main hydrocarbon product, which are highly undesirable. This is in stark contrast to the catalyst composition of Example B1, which gave CO conversions ranging from 20 to 55% under the same conditions. It is likely that during the syngas conversion process, some of the oxides in the comparative catalyst composition may have been reduced to lower oxidative state including metallic state. However, it is believed that carbide and/or nitride phase is unlikely to form during the syngas conversion process in the comparative catalyst composition. On the contrary, in the inventive catalyst composition of Example B1, both metal carbides and metal nitrides are present and are believed to be intimately mixed with each other and other phases including individual metal phases, mixed metal phases, carbon phase, carbon nitride phases, and the like, providing high catalytic activity to the catalyst composition.

### Examples B4

A series of catalyst precursors of this disclosure fabricated according to the procedures similar to those of Examples A1 and A2 above were further thermally decomposed to form exemplary catalytic components and exemplary catalyst compositions in a syngas conversion reactor similar to that described in Example B1 above. The in-situ formed catalyst compositions were then tested for performance in exemplary syngas conversion processes in the syngas reactor under conversion conditions similar to those in Example B1 above. The catalytic components of all these catalyst compositions contain carbon and nitrogen, at least part of which in the form of metal carbide(s) and/or metal nitride(s). The metal elements contained in these catalytic components are indicated in the following TABLE V (B4(iii) being the same as the catalytic component in Example B2), along with several performance parameters at 250° C., 2:1 H₂/CO ratio, absolute pressure of 1800 kPa, GHSV=2000 h⁻¹:

TABLE V

| Example No. | B4 (i) | B4 (ii) | B4 (iii) | B4 (iv) | B4 (v) | B4 (vi) |
|---|---|---|---|---|---|---|
| Metal Elements | Co-Gd | Fe-Gd | Co-Mn-Y | Co-Y | Co-Cu-Pr | Co-La |
| CO Conversion (%) | 14 | 19 | 27 | 11 | 15 | 33 |
| C₂-C₄ products (wt %) | 27 | 21 | 37 | 30 | 36 | 27 |
| Olefins in C₂-C₄ products (wt %) | 78 | 77 | 61 | 54 | 67 | 57 |
| Chemicals (Olefins + Alcohols) | 78 | 88 | 76 | 68 | 79 | 77 |
| in C₂-C₄ products (wt %) | | | | | | |

As can be seen from the data in TABLE V above, this category of catalyst compositions, bimetallic or trimetallic, comprising metal carbide(s) and/or metal nitride(s), demonstrated high activity in converting syngas into organic products, especially C2-C4 olefins and C2-C4 alcohols, which have significantly higher value than syngas. Moreover, these catalysts are highly selective toward C2-C4 olefins and alcohols, and particularly toward C2-C4 olefins, among all C2-C4 products produced. Overall, the C2-C4 product fraction comprises 50-80% olefins and 10-20% alcohols.

Other non-limiting aspects and/or embodiments of the present disclosure can include:

A1. A catalyst composition for converting syngas comprising a catalytic component, wherein the catalytic component comprises:

a metal element M¹, selected from iron, cobalt, manganese, and combinations of two or more thereof at any proportion;

a metal element M², selected from aluminum, gallium, indium, thallium, chromium, scandium, yttrium, the lanthanide series, the actinide series, and any combination of two or more thereof at any proportion;

an optional metal M³, differing from M¹ and M²;

carbon;

nitrogen; and

optionally sulfur, at a molar ratio of M², M³, carbon, nitrogen, and sulfur to M¹ of r1, r2, r3, r4, and r5, respectively, indicated below:

M²:M³:C:N:S:M¹=r1:r2:r3:r4:r5:1, where:

0.1≤r1≤1.5;

0≤r2≤0.5;

0<r3≤1;

0<r4≤1; and

0≤r5≤1.

A2. The catalyst composition of embodiment A1, wherein 0.1≤r1≤1.0, preferably 0.5≤r1≤1.0.

A3. The catalyst composition of embodiment A1 or A2, wherein at least a portion of the carbon in the catalytic component is present as a metal carbide of one or more of M¹, M², and M³, and/or at least a portion of the nitrogen in the catalytic component is present as a metal nitride of one or more of M¹, M², and M³, as determined by x-ray diffraction diagram of the catalytic component.

A4. The catalyst composition of any of embodiments A1 to A3, wherein at least a portion of the carbon in the catalytic component is present as a metal carbide of one or more of M¹ and M², and/or at least a portion of the nitrogen in the catalytic component is present as a metal nitride of one of more of M¹ and M², as determined by x-ray diffraction diagram of the catalytic component.

A4a. The catalyst composition of embodiment A4, wherein at least a portion of the carbon in the catalytic component is present as a metal carbide of one or both of iron and cobalt, and/or at least a portion of the nitrogen in the catalytic component is present as a metal nitride of one or more of iron or cobalt.

A4b. The catalyst composition of embodiment A4 or A4a, wherein the metal carbide and/or the metal nitride are distributed homogenously in the catalytic component.

A5. The catalyst composition of any of embodiments A1 to A4b, wherein M¹ is selected from iron, cobalt, combinations of iron and cobalt at any proportion, combinations of iron and manganese at any proportion, combination of cobalt with manganese at any proportion, and combination of iron, cobalt, and manganese at any proportion.

A6. The catalyst composition of any of embodiments A1 to A5, wherein M² is selected from aluminum, gallium, indium, thallium, chromium, scandium, yttrium, and the lanthanide series.

A7. The catalyst composition of any of embodiments A1 to A6, wherein M³ is selected from alkali metals, copper, silver, and any combinations and mixtures of two or more thereof at any proportion.

A8. The catalyst composition of any of embodiments A1 to A7, wherein the catalytic component consists essentially of M¹, M², M³, carbon, nitrogen, and optionally sulfur; e.g., comprising ≥85, or ≥90, or ≥95, or ≥98, or even ≥99 wt % of M¹, M², M³, carbon, nitrogen, and optionally sulfur, based on the total weight of the catalytic component.

A9. The catalyst composition of any of embodiments A1 to A8, wherein r1≈1.0.

A10. The catalyst composition of any of embodiments A1 to A9, wherein 0≤r5≤0.5.

A11. The catalyst composition of any of embodiments A1 to A10, further comprising a support.

B1. A catalyst composition comprising a catalytic component, wherein the catalytic component comprises:

a metal element M¹, selected from iron, cobalt, manganese, and combinations of two or more thereof at any proportion;

a metal element M², selected from aluminum, gallium, indium, thallium, scandium, yttrium, the lanthanide series, the actinide series, and any combination of two or more thereof at any proportion;

an optional metal M³, differing from M¹ and M²;

carbon;

nitrogen; and

optionally sulfur, and wherein:

at least a portion of the carbon in the catalytic component is present as a metal carbide of one or more of M¹, M², and M³, and/or at least a portion of the nitrogen in the catalytic component is present as a metal nitride of one or more of M¹, M², and M³, as determined by x-ray diffraction diagram of the catalytic component.

B2. The catalyst composition of embodiment B1, wherein at least a portion of the carbon in the catalytic component is present as a metal carbide of one or more of M¹ and M², and/or at least a portion of the nitrogen in the catalytic component is present as a metal nitride of one of more of M¹ and M², as determined by x-ray diffraction diagram of the catalytic component.

B3. The catalyst composition of embodiment B1 or B2, wherein at least a portion of the carbon in the catalytic component is present as a metal carbide of iron or cobalt, and/or at least a portion of the nitrogen in the catalytic component is present as a metal nitride of one or more of iron or cobalt.

B3a. The catalyst composition of any of embodiments B1 to B3, wherein the metal carbide and/or the metal nitride are distributed homogenously in the catalytic component.

B4. The catalyst composition of any of embodiments B1 to B3a, wherein the catalytic component has a molar ratio of M², M³, carbon, nitrogen, and sulfur to M¹ of r1, r2, r3, r4, and r5, respectively, indicated below:

M²:M³:C:N:S:M¹=r1:r2:r3:r4:r5:1, where:

0.1≤r1≤1.5;

0≤r2≤0.5;

0<r3≤1;

0<r4≤1; and

0≤r5≤1.

B5. The catalyst composition of embodiment B4, wherein 0.1≤r1≤1.0, preferably 0.5≤r1≤1.0.

B6. The catalyst composition of any of embodiments B1 to B5, wherein M¹ is selected from iron, cobalt, combinations of iron and cobalt at any proportion, combinations of iron and manganese at any proportion, combination of cobalt with manganese at any proportion, and combination of iron, cobalt, and manganese at any proportion.

B7. The catalyst composition of any of embodiments B1 to B6, wherein M² is selected from aluminum, gallium, indium, thallium, chromium, scandium, yttrium, and the lanthanide series.

B8. The catalyst composition of any of embodiments B1 to B7, wherein M³ is selected from alkali metals, copper, silver, and any combinations and mixtures of two or more thereof at any proportion.

B9. The catalyst composition of any of embodiments B1 to B8, wherein the catalytic component consists essentially of M¹, M², M³, carbon, nitrogen, and optionally sulfur, e.g., comprising ≥85, or ≥90, or ≥95, or ≥98, or even ≥99 wt % of M¹, M², M³, carbon, nitrogen, and optionally sulfur, based on the total weight of the catalytic component.

B10. The catalyst composition of any of embodiments B1 to B9, wherein r1≈1.0.

B11. The catalyst composition of any of embodiments B1 to B10, wherein 0≤r5≤0.5.

B12. The catalyst composition of any of embodiments B1 to B11, further comprising a support.

C1. A catalyst precursor of a catalyst, comprising a first precursor component having the following formula (F-PM-1), a second precursor component having the following formula (F-PM-2), or a mixture or combination of both the first precursor component and the second precursor component:

M^{b}_{q-p}(M^{a}L_{q})ₘ (F-PM-1)

M^{b}Lₘ (F-PM-2)

where M^{a} is a metal element in +p valency selected from manganese, iron, cobalt, and combinations and mixtures of two or more thereof at any proportion, L, the same or different at each occurrence, is a ligand selected from CN⁻, OCN⁻, and SCN⁻, in (F-PM-1), M^{a} complexes with q units of L on average to form a complex anion in p-q average valency, M^{b} is a metal element selected from aluminum, gallium, indium, thallium, iron, cobalt, chromium, manganese, scandium, yttrium, the lanthanide series, the actinide series, and any combination or mixture of two or more thereof at any proportion, providing a cation in +m valency, where p is 2, 3, 4, or 5, 2≤q≤6, and m is 2, 3, 4, 5, or 6.

C2. The catalyst precursor of embodiment C1, comprising at least two of the first precursor component having formula (F-PM-1) above, wherein at least one such first precursor component comprises iron as M^{a}, and at least one other such first precursor component comprises manganese as M^{a}.

C3. The catalyst precursor of embodiment C2, comprising at least two precursor materials having formula (F-PM-1) above, wherein at least one such precursor material comprises cobalt as M^{a}, and at least one other precursor material comprises manganese as M^{a}.

C4. The catalyst precursor of any of embodiments C1 to C3, wherein M^{b} is selected from iron, cobalt, manganese, scandium, yttrium, the lanthanide series, and combinations of at least two thereof.

C5. The catalyst precursor of any of embodiments C1 to C5, wherein m is 3, and q-p is 3.

C6. The catalyst precursor of any of embodiments C1 to C6, wherein the precursor material is selected from the following:

ME(Co(III)L₆)

ME(Fe(III)L₆)

where ME is a metal element in +3 valency selected from iron, cobalt, manganese, scandium, yttrium, the lanthanide series, and combinations of at least two thereof.

C7. The catalyst precursor of any of embodiments C1 to C6, which is insoluble in water at room temperature.

C9. The catalyst precursor of embodiment C7, which consists essentially of the first precursor component, e.g., comprising ≥85, or ≥90, or ≥95, or ≥98, or even ≥99 wt % of the first precursor component, based on the total weight of the catalyst precursor.

C10. The catalyst precursor of embodiment C7, which consists essentially of the second precursor component, e.g., comprising ≥85, or ≥90, or ≥95, or ≥98, or even ≥99 wt % of the second precursor component, based on the total weight of the catalyst precursor.

C11. The catalyst precursor of any of embodiments C1 to C10, further comprising a catalyst support material.

C12. The catalyst precursor of any of embodiments C1 to C11, further comprising a precursor of a catalyst support material.

D1. A process for making a catalytic composition, the process comprising:

(i) providing a first material comprising a first compound having the following formula (F-I-1), and/or a second compound having the following formula (F-1-2), or a mixture of the first compound and the second compound:

M^{d}_{q-p}(M^{a}L_{q})ₖ (F-I-1)

M^{e}Lₓ (F-I-2)

where M^{a} is a metal element in +p valency selected from manganese, iron, cobalt, and combinations and mixtures of two or more thereof at any proportion, L, the same or different at each occurrence, is a ligand selected from CN⁻, OCN⁻, and SCN⁻, M^{a} complexes with q units of L on average to form a complex anion in p-q average valency, M^{d} is a metal element or a group providing a cation in +k valency, and M^{e} is a metal element or a group providing a cation in +x valency, where p is 2, 3, 4, or 5, 2≤q≤6, k is 1, 2, 3, 4, 5, or 6, and x is 1, 2, 3, 4, 5, or 6;

(ii) providing a second material having the following formula (F-II):

M^{b}ₙAₘ (F-II)

where M^{b} is a metal element in +m valency selected from aluminum, gallium, indium, thallium, iron, cobalt, chromium, manganese, scandium, yttrium, the lanthanide series, the actinide series, and any combination or mixture of two or more thereof at any proportion, and A is an anion in −n valency, wherein A differs from the complex anion in (F-I), m is 2, 3, 4, 5, or 6, and n is 1, 2, 3, 4, 5, or 6; and

(iii) reacting the first material and the second material to obtain a first solid precursor comprising first precursor component having the following formula (F-PM-1), or a second precursor component having the following formula (F-PM-2), or a mixture or combination of both the first precursor component and the second precursor component:

M^{b}_{q-p}(M^{a}L_{q})ₘ (F-PM-1)

M^{b}Lⱼ (F-PM-2)

where j is an integer or non-integer, and m−1≤j≤m.

D1a. The process of embodiment D1a, wherein the first compound and the second compound are water soluble at a temperature in the range from 20 to 80° C., preferably water soluble at room temperature.

D1b. The process of embodiment D1 or D1a, wherein the reacting step (iii) is conducted in a liquid dispersion.

D2. The process of embodiment D1, D1a, or D1b, further comprising:

(iv) adding a third material comprising a metal element M^{c}, to the first solid precursor to obtain a second solid precursor.

D3. The process of embodiment D2, wherein step (iv) is effected at least partly simultaneously in step (iii), wherein the first material, the second material and the third material are combined, and after step (iii), the first solid precursor is separated from a liquid phase in the liquid dispersion, and the first solid precursor carries a quantity of the third material.

D4. The process of embodiment D2 or D3, wherein step (iv) is performed at least partly after step (iii), and the process further comprises:

(iiia) after step (iii), separating the first solid precursor from a liquid phase in the liquid dispersion;

(iiib) optionally washing the separated first solid precursor using a solvent; and subsequently

(iiic) impregnating the separated first solid precursor with a dispersion of the third material in a liquid.

D4a. The process of embodiment D4, further comprising:

(iiid) after step (iiic), drying and/or calcining the impregnated first solid precursor to obtain the second solid precursor.

D4b. The process of any of embodiments D4, D4, or D4a, wherein the third material comprises a water soluble compound of M^{c}.

D5c. The process of embodiment D4b, wherein the third material comprises a nitrate, a nitrite, a chloride, a fluoride, a bromide, an acetate, a citrate, of M^{c}, or a mixture or combination of two or more thereof at any proportion.

D5. The process of any of embodiments D1 to D4, wherein M^{d} and M^{e} are independently selected from an alkali metal, an ammonium group, and a proton.

D6. The process of any of embodiments D1 to D5, wherein the liquid dispersion in step (iii) comprises water.

D7. The process of any of embodiments D4 to D6, wherein the solvent in step (iiib) comprises water.

D8. The process of claim D6, wherein the liquid dispersion comprises water as the sole solvent for the first material and the second material.

D9. The process of claim D6, wherein an aqueous dispersion of the first material is mixed with another aqueous dispersion of the second material to effect step (iii).

D10. The process of embodiment D9, wherein an aqueous solution of the first material is mixed with another aqueous solution of the second material to effect step (iii).

D11. The process of any of embodiments D1 to D9, wherein m=3, and q-p=3.

D12. The process of any of embodiments D1 to D11, further comprising:

(iva) combining the first solid precursor and/or the second solid precursor with a catalyst support material.

D13. The process of any of embodiments D1 to D12, further comprising:

(v) heating the first solid precursor and/or the second solid precursor at a temperature of at least 200° C. in the presence of an inert atmosphere for a period of at least 1 minute to obtain a catalytic component.

D14. The process of embodiment D13, wherein in step (v), the temperature is no higher than 800° C.

D15. The process of embodiments D13 or D14, wherein the catalytic component comprises M^{a}, M^{b}, optionally M^{c}, carbon, nitrogen, and optionally sulfur, at a molar ratio of M^{b}, M^{c}, carbon, nitrogen, and sulfur to M^{a} of r1, r2, r3, r4, and r5, respectively, indicated below:

M^{b}:M^{c}:C:N:S:M^{a}=r1:r2:r3:r4:r5:1, where:

0.1≤r1≤1.5;

0≤r2≤0.5;

0<r3≤1;

0<r4≤1; and

0≤r5≤1.

where M^{a} is selected from manganese, iron, cobalt, and combinations and mixtures of two or more thereof at any proportion, M^{b} is selected from aluminum, gallium, indium, thallium, chromium, scandium, yttrium, the lanthanide series, the actinide series, and any combination or mixture of two or more thereof at any proportion, M^{c} is any metal different from M^{a} and M^{b}.

D16. The ^{process} of embodiment D15, wherein M^{c} is selected from alkali metals, copper, silver, and any combinations or mixtures of two or more thereof at any proportion.

D17. The process of any of embodiment D13 to D16, wherein at least a portion of the carbon in the catalytic component is present as a metal carbide of one or more of M^{a}, M^{b}, and M^{c}, and/or at least a portion of the nitrogen in the catalytic component is present as a metal nitride of one or more of M^{a}, M^{b}, and M^{c}, as determined by x-ray diffraction diagram of the catalytic component.

D18. The process of embodiment D17, wherein at least a portion of the carbon in the catalytic component is present as a metal carbide of one or more of M¹ and M², and/or at least a portion of the nitrogen in the catalytic component is present as a metal nitride of one of more of M¹ and M², as determined by x-ray diffraction diagram of the catalytic component.

D19. The process of any of embodiment D17 or D18, wherein the metal carbide and/or the metal nitride are distributed homogeneously in the catalytic component.

E1. A process for converting syngas, the process comprising contacting a feed comprising syngas with a catalyst composition of any of embodiments A1 to A11 in a conversion reactor to produce a conversion product mixture.

E2. The process of embodiment E1, wherein the feed consists essentially of syngas, e.g., comprising ≥85, or ≥90, or ≥95, or ≥98, or even ≥99 wt % of syngas, based on the total weight of the feed.

E3. The process of embodiment E1 or E2, wherein the conversion product comprises at least one C2-C5 olefin and/or at least one C1-C5 alcohol.

F1. A process for converting syngas, the process comprising contacting a feed comprising syngas with a catalyst composition of any of embodiments B1 to B12 in a conversion reactor to produce a conversion product mixture.

F2. The process of embodiment F1, wherein the feed consists essentially of syngas, e.g., comprising ≥85, or ≥90, or ≥95, or ≥98, or even ≥99 wt % of syngas, based on the total weight of the feed.

F3. The process of embodiment F1 or F2, wherein the conversion product comprises at least one C2-C5 olefin and/or at least one C1-C5 alcohol.

G1. A process for converting syngas, the process comprising:

(A) disposing a catalyst precursor of any of embodiments C1 to C6 in a conversion reactor;

(B) heating the catalyst precursor in the conversion reactor at a temperature of at least 200° C., preferably in a range from 200 to 800° C., more preferably in a range from 200 to 600° C., still more preferably in a range from 300 to 500° C., in the presence of an inert atmosphere for a period of at least 1 minute to obtain a catalytic component; and

(C) contacting the catalytic component with a feed comprising syngas in the conversion reactor under conversion conditions effective to convert syngas to produce a conversion product mixture.

G2. The process of embodiment G1, wherein the conversion product mixture comprises at least one of a C2 to C5 (such as C2 to C4) olefin and/or at least one of a C1 to C5 (such as C1 to C4) alcohol.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents described herein are incorporated by reference herein, including any priority documents and/or testing procedures to the extent they are not inconsistent with this text. As is apparent from the foregoing general description and the specific embodiments, while forms of this disclosure have been illustrated and described, various modifications can be made without departing from the spirit and scope of this disclosure. Accordingly, it is not intended that this disclosure be limited thereby. Likewise, the term “comprising” is considered synonymous with the term “including” for purposes of United States law. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

While this disclosure has been described with respect to a number of embodiments and examples, those skilled in the art, having benefit of this disclosure, will appreciate that other embodiments can be devised which do not depart from the scope and spirit of this disclosure.

1. A catalyst composition comprising a catalytic component, wherein the catalytic component comprises:
a metal element M¹, selected from iron, cobalt, manganese, and combinations of two or more thereof at any proportion;
a metal element M², selected from aluminum, gallium, indium, thallium, chromium, scandium, yttrium, the lanthanide series, the actinide series, and any combination of two or more thereof at any proportion;
an optional metal M³, differing from M¹ and M²;
carbon;
nitrogen; and
optionally sulfur, at a molar ratio of M², M³, carbon, nitrogen, and sulfur to M¹ of r1, r2, r3, r4, and r5, respectively, indicated below:
M²:M³:C:N:S:M¹=r1:r2:r3:r4:r5:1, where:
0.1≤r1≤1.5;
0≤r2≤0.5;
0<r3≤1;
0<r4≤1; and
0≤r5≤1.

2. The catalyst composition of claim 1, wherein at least a portion of the carbon in the catalytic component is present as a metal carbide of one or more of M¹, M², and M³, and at least a portion of the nitrogen in the catalytic component is present as a metal nitride of one or more of M¹, M², and M³, as determined by x-ray diffraction diagram of the catalytic component.

3. The catalyst composition of claim 2, wherein the metal carbide and/or the metal nitride are distributed homogenously in the catalytic component.

4. The catalyst composition of claim 1, wherein M¹ is selected from iron, cobalt, combinations of iron and cobalt at any proportion, combinations of iron and manganese at any proportion, combination of cobalt with manganese at any proportion, and combination of iron, cobalt, and manganese at any proportion.

5. The catalyst composition of claim 1, wherein M² is selected from yttrium and the lanthanide series.

6. The catalyst composition of claim 1, wherein M³ is selected from alkali metals, copper, silver, and any combinations and mixtures of two or more thereof at any proportion.

7. The catalyst composition of claim 1, wherein the catalytic component consists essentially of M¹, M², M³, carbon, nitrogen, and optionally sulfur.

8. The catalyst composition of claim 1, wherein r1 is a number in the range from 0.9 to 1.1.

9. A catalyst composition comprising a catalytic component, wherein the catalytic component comprises:
a metal element M¹, selected from iron, cobalt, manganese, and combinations of two or more thereof at any proportion;
a metal element M², selected from aluminum, gallium, indium, thallium, chromium, scandium, yttrium, the lanthanide series, the actinide series, and any combination of two or more thereof at any proportion;
an optional metal M³, differing from M¹ and M²;
carbon;
nitrogen; and
optionally sulfur, and wherein:
at least a portion of the carbon in the catalytic component is present as a metal carbide of one or more of M¹, M², and M³, and at least a portion of the nitrogen in the catalytic component is present as a metal nitride of one or more of M¹, M², and M³, as determined by x-ray diffraction diagram of the catalytic component.

10. The catalyst composition of claim 9, wherein the metal carbide and/or the metal nitride are distributed homogenously in the catalytic component.

11. The catalyst composition of claim 9, wherein the catalytic component has a molar ratio of M², M³, carbon, nitrogen, and sulfur to M¹ of r1, r2, r3, r4, and r5, respectively, indicated below:
M²:M³:C:N:S:M¹=r1:r2:r3:r4:r5:1, where:
0.1≤r1≤1.5;
0≤r2≤0.5;
0<r3≤1;
0<r4≤1; and
0≤r5≤1.

12. A catalyst precursor of a catalyst, comprising a first precursor component having the following formula (F-PM-1), or a second precursor component having the following formula (F-PM-2), or a mixture or combination of both the first precursor component and the second precursor component:
M^{b}_{q-p}(M^{a}L_{q})ₘ (F-PM-1)
M^{b}Lⱼ (F-PM-2)
where M^{a} is a metal element in +p valency selected from manganese, iron, cobalt, and combinations and mixtures of two or more thereof at any proportion, L, the same or different at each occurrence, is a ligand selected from CN⁻, OCN⁻, and SCN⁻, M^{a} complexes with q units of L on average to form a complex anion in p-q average valency, M^{b} is a metal element selected from aluminum, gallium, indium, thallium, iron, cobalt, chromium, manganese, scandium, yttrium, the lanthanide series, the actinide series, and any combination or mixture of two or more thereof at any proportion, providing a cation in +m valency, where j is an integer or non-integer, and m−1≤j≤m, m is 2, 3, 4, 5, or 6, p is 2, 3, 4, or 5, q is an integer or non-integer, and 2≤q≤6.

13. The catalyst precursor of claim 12, comprising at least two of the first precursor component having formula (F-PM-1) above, wherein at least one such first precursor component comprises iron as M^{a}, and at least one other precursor material comprises manganese as M^{a}.

14. The catalyst precursor of claim 12, comprising at least two of the first precursor component having formula (F-PM-1) above, wherein at least one such first precursor component comprises cobalt as M^{a}, and at least one other precursor material comprises manganese as M^{a}.

15. A process for making a catalytic composition, the process comprising:
(i) providing a first material comprising a first compound having the following formula (F-I-1), and/or a second compound having the following formula (F-1-2), or a mixture of the first compound and the second compound:
M^{d}_{q-p}(M^{a}L_{q})ₖ (F-I-1)
M^{e}Lₓ (F-I-2)
where M^{a} is a metal element in +p valency selected from manganese, iron, cobalt, and combinations and mixtures of two or more thereof at any proportion, L, the same or different at each occurrence, is a ligand selected from CN⁻, OCN⁻, and SCN⁻, M^{a} complexes with q units of L on average to form a complex anion in p-q average valency, M^{d} is a metal element or a group providing a cation in +k valency, and M^{c} is a metal element or a group providing a cation in +x valency, where p is 2, 3, 4, or 5, q is an integer or non-integer, 2≤q≤6, k is 1, 2, 3, 4, 5, or 6, and x is 1, 2, 3, 4, 5, or 6;
(ii) providing a second material having the following formula (F-II):
M^{b}ₙAₘ (F-II)
where M^{b} is a metal element in +m valency selected from aluminum, gallium, indium, thallium, iron, cobalt, chromium, manganese, scandium, yttrium, the lanthanide series, the actinide series, and any combination or mixture of two or more thereof at any proportion, and A is an anion in −n valency, wherein A differs from the complex anion in (F-I), m is 2, 3, 4, 5, or 6, and n is 1, 2, 3, 4, 5, or 6; and
(iii) reacting the first material and the second material to obtain a first solid precursor comprising first precursor component having the following formula (F-PM-1), or a second precursor component having the following formula (F-PM-2), or a mixture or combination of both the first precursor component and the second precursor component:
M^{b}_{q-p}(M^{a}L_{q})ₘ (F-PM-1)
M^{b}Lⱼ (F-PM-2)
where j is an integer or non-integer, and m−1≤j≤m.

16. The process of claim 15, further comprising:
(iv) adding a third material comprising a metal element M^{c}, to the first solid precursor to obtain a second solid precursor.

17. The process of claim 15, further comprising:
(v) heating the first solid precursor and/or the second solid precursor at a temperature of at least 200° C. in the presence of an inert atmosphere for a period of at least 1 minute to obtain a catalytic component.

18. The process of claim 16, wherein step (iv) comprises impregnating the first solid precursor with a liquid dispersion comprising the third material to obtain a solid/liquid mixture, followed by drying the solid/liquid mixture to obtain the second solid precursor.

19. The process of claim 17, wherein the catalytic component comprises M^{a}, M^{b}, optionally M^{c}, carbon, nitrogen, and optionally sulfur, at a molar ratio of M^{b}, M^{c}, carbon, nitrogen, and sulfur to M^{a} of r1, r2, r3, r4, and r5, respectively, indicated below:
M^{b}:M^{c}:C:N:S:M^{a}=r1:r2:r3:r4:r5:1, where:
0.1≤r1≤1.5;
0≤r2≤0.5;
0<r3≤1;
0<r4≤1; and
0≤r5≤1.
where M^{a} is selected from manganese, iron, cobalt, and combinations and mixtures of two or more thereof at any proportion, M^{b} is selected from aluminum, gallium, indium, thallium, chromium, scandium, yttrium, the lanthanide series, the actinide series, and any combination or mixture of two or more thereof at any proportion, M^{c} is selected from alkali metals, copper, silver, and any combinations or mixtures of two or more thereof at any proportion.

20. The process of claim 17, wherein at least a portion of the carbon in the catalytic component is present as a metal carbide of one or more of M^{a}, M^{b}, and M^{c}, and/or at least a portion of the nitrogen in the catalytic component is present as a metal nitride of one or more of M^{a}, M^{b}, and M^{c}, as determined by x-ray diffraction diagram of the catalytic component.

21. The process of claim 20, wherein the metal carbide and/or the metal nitride are distributed homogenously in the catalytic component.

22. A process for converting syngas, the process comprising contacting a feed comprising syngas with a catalyst composition of claim 1 in a conversion reactor to produce a conversion product mixture.

23. A process for converting syngas, the process comprising contacting a feed comprising syngas with a catalyst composition of claim 1 in a conversion reactor to produce a conversion product mixture.

24. A process for converting syngas, the process comprising:
(A) disposing a catalyst precursor of claim 13 in a conversion reactor;
(B) heating the catalyst precursor in the conversion reactor at a temperature of at least 200° C. in the presence of an inert atmosphere for a period of at least 1 minute to obtain a catalytic component; and
(C) contacting the catalytic component with a feed comprising syngas under conversion conditions effective to convert syngas to a conversion product mixture.

25. The process of claim 24, wherein the conversion product mixture comprises at least one of a C2-C5 olefin and/or at least one of a C1-C5 alcohol.
